SEQUENCE LISTING (74 sequences)

SEQ ID NO: 1 - protein, 90 aa, Homo sapiens
SYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 2 - protein, 91 aa, Synthetic construct
CSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 3 - protein, 90 aa, Mus musculus
SYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 4 - protein, 78 aa, Synthetic construct
FKDPVADPNKRSKKGRLSLHRTPAGNFVTLEEGKGDLEEYGQDLLHTVFKNGKVTKSYSFDEIRKNAQLNIELEAAHH

SEQ ID NO: 5 - protein, 93 aa, Synthetic construct
HHHHHHHHENLYFQGFKDPVADPNKRSKKGRLSLHRTPAGNFVTLEEGKGDLEEYGQDLLHTVFKNGKVTKSYSFDEIRKNAQLNIELEAAHH

SEQ ID NO: 6 - protein, 70 aa, Synthetic construct
NKRSKKGRLSLHRTPAGNFVTLEEGKGDLEEYGQDLLHTVFKNGKVTKSYSFDEIRKNAQLNIELEAAHH

SEQ ID NO: 7 - protein, 85 aa, Synthetic construct
HHHHHHHHENLYFQGNKRSKKGRLSLHRTPAGNFVTLEEGKGDLEEYGQDLLHTVFKNGKVTKSYSFDEIRKNAQLNIELEAAHH

SEQ ID NO: 8 - protein, 62 aa, Synthetic construct
LSLHRTPAGNFVTLEEGKGDLEEYGQDLLHTVFKNGKVTKSYSFDEIRKNAQLNIELEAAHH

SEQ ID NO: 9 - protein, 77 aa, Synthetic construct
HHHHHHHHENLYFQGLSLHRTPAGNFVTLEEGKGDLEEYGQDLLHTVFKNGKVTKSYSFDEIRKNAQLNIELEAAHH

SEQ ID NO: 10 - protein, 56 aa, Synthetic construct
PAGNFVTLEEGKGDLEEYGQDLLHTVFKNGKVTKSYSFDEIRKNAQLNIELEAAHH

SEQ ID NO: 11 - protein, 71 aa, Synthetic construct
HHHHHHHHENLYFQGPAGNFVTLEEGKGDLEEYGQDLLHTVFKNGKVTKSYSFDEIRKNAQLNIELEAAHH

SEQ ID NO: 12 - protein, 19 aa, Synthetic construct
RRRPKGRGKRRREKQRPTD

SEQ ID NO: 13 - protein, 13 aa, Synthetic construct
RKKKGGKNGKNRR

SEQ ID NO: 14 - protein, 10 aa, Synthetic construct
WREPSFMALS

SEQ ID NO: 15 - protein, 196 aa, Synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 16 - protein, 7 aa, Synthetic construct
TKKTLRT

SEQ ID NO: 17 - protein, 110 aa, Synthetic construct
RRRPKGRGKRRREKQRPTDCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 18 - protein, 104 aa, Synthetic construct
RKKKGGKNGKNRRCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 19 - protein, 491 aa, Homo sapiens [sequence omitted]

SEQ ID NO: 20 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 21 - DNA, 272 bp, Synthetic construct
  source 1..272: mol_type = other DNA
agctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcatta

SEQ ID NO: 22 - DNA, 271 bp, Synthetic construct
  source 1..271: mol_type = other DNA
agctatgttgtaaccaatggccttggggttaatgtgtttaaggacccagttgctgatcccaacaaaaggtcaaaaaagggccggttatctttacataggacaccagcggggaactttgttacacttgaagaaggaaaaggagaccttgaggaatatggccatgatcttctccatacggttttcaagaatgggaaggtgacaaaaagctactcatttgatgaagtcagaaaaaatgcacagctgaacatcgagcaggacgtggcacctcatt

SEQ ID NO: 23 - DNA, 1475 bp, Synthetic construct
  source 1..1475: mol_type = other DNA
atgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcatta

SEQ ID NO: 24 - DNA, 1474 bp, Synthetic construct
  source 1..1474: mol_type = other DNA
atgaatgctgcggcagaagccgagttcaacatcctgctggccaccgactcgtacaaggttactcactataaacaatacccacccaacacaagcaaagtttattcctactttgaatgccgtgaaaagaagacagaaaactccaaagtaaggaaggtgaaatacgaggaaacagtattttatgggttgcagtacattcttaataagtacttaaaaggtaaagtagtgaccaaagagaaaatccaggaggccaaagaagtgtacagagaacatttccaagatgatgtctttaacgaaagaggatggaactacatccttgagaaatacgatggtcatctcccgattgaagtaaaggctgttcccgagggctctgtcatccccagagggaacgtgctgttcacagtggaaaacacagacccagagtgctactggcttaccaattggattgagactattcttgttcagtcctggtatccaattacagtggccacaaattccagagaacagaagagaatactggccaaatatttgttagaaacctctggtaacttagatggtctggaatacaagttacatgactctggttacagaggagtctcttcgcaagagactgctggcataggggcatctgctcatttggttaacttaaaaggaacagatactgtggcgggaattgctctaattaaaaaatactatgggacaaaagatcctgttccaggctattctgttccagcagcagagcacagtaccataacggcttgggggaaagaccatgagaaagatgcttttgaacacatagtaacacagttctcatcagtgcctgtgtctgtggtcagcgatagctatgacatttataatgcgtgtgagaaaatatggggtgaagacctgagacatctgatagtatcgagaagtacagaggcaccactaatcatcagacctgactctggaaatcctcttgacactgtattgaaggtcttagatattttaggcaagaagtttcctgttactgagaactcaaaaggctacaagttgctgccaccttatcttagagtcattcaaggagatggcgtggatatcaatactttacaagagattgtagagggaatgaaacaaaagaagtggagtatcgagaatgtctccttcggttctggtggcgctttgctacagaagttaacccgagacctcttgaattgctccttcaagtgcagctatgttgtaaccaatggccttggggttaatgtgtttaaggacccagttgctgatcccaacaaaaggtcaaaaaagggccggttatctttacataggacaccagcggggaactttgttacacttgaagaaggaaaaggagaccttgaggaatatggccatgatcttctccatacggttttcaagaatgggaaggtgacaaaaagctactcatttgatgaagtcagaaaaaatgcacagctgaacatcgagcaggacgtggcacctcatt

SEQ ID NO: 25 - DNA, 237 bp, Synthetic construct
  source 1..237: mol_type = other DNA
ttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 26 - DNA, 213 bp, Synthetic construct
  source 1..213: mol_type = other DNA
aacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 27 - DNA, 189 bp, Synthetic construct
  source 1..189: mol_type = other DNA
ctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 28 - DNA, 171 bp, Synthetic construct
  source 1..171: mol_type = other DNA
ccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 29 - DNA, 303 bp, Synthetic construct
  source 1..303: mol_type = other DNA
tggcgcgaaccgagctttatggcgctgagctcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 30 - DNA, 861 bp, Synthetic construct
  source 1..861: mol_type = other DNA
tgcagccagccgctggatgtgattctgctgctggatggcagcagcagctttccggcgagctattttgatgaaatgaaaagctttgcgaaagcgtttattagcaaagcgaacattggcccgcgcctgacccaggtgagcgtgctgcagtatggcagcattaccaccattgatgtgccgtggaacgtggtgccggaaaaagcgcatctgctgagcctggtggatgtgatgcagcgcgaaggcggcccgagccagattggcgatgcgctgggctttgcggtgcgctatctgaccagcgaaatgcatggcgcgcgcccgggcgcgagcaaagcggtggtgattctggtgaccgatgtgagcgtggatagcgtggatgcggcggcggatgcggcgcgcagcaaccgcgtgaccgtgtttccgattggcattggcgatcgctatgatgcggcgcagctgcgcattctggcgggcccggcgggcgatagcaacgtggtgaaactgcagcgcattgaagatctgccgaccatggtgaccctgggcaacagctttctgcataaactgtgcagcggctttgtgcgcatttgcaccggctcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 31 - DNA, 294 bp, Synthetic construct
  source 1..294: mol_type = other DNA
accaaaaaaaccctgcgcacctcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 32 - DNA, 273 bp, Synthetic construct
  source 1..273: mol_type = other DNA
tcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 33 - protein, 49 aa, Synthetic construct
NKRSKKGRLSLHRTPAGNFVTLEEGKGDLEEYGQDLLHTVFKNGKVTKS

SEQ ID NO: 34 - protein, 64 aa, Synthetic construct
HHHHHHHHENLYFQGNKRSKKGRLSLHRTPAGNFVTLEEGKGDLEEYGQDLLHTVFKNGKVTKS

SEQ ID NO: 35 - DNA, 150 bp, Synthetic construct
  source 1..150: mol_type = other DNA
aacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttac

SEQ ID NO: 36 - protein, 20 aa, Synthetic construct
MYRMQLLSCIALSLALVTNS

SEQ ID NO: 37 - protein, 242 aa, Synthetic construct
MYRMQLLSCIALSLALVTNSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 222 aa, Synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 242 aa, Synthetic construct
MYRMQLLSCIALSLALVTNSCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 222 aa, Synthetic construct
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 15 aa, Synthetic construct
GGGGSGGGGSGGGGS

SEQ ID NO: 42 - protein, 307 aa, Synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNKRSKKGRLSLHRTPAGNFVTLEEGKGDLEEYGQDLLHTVFKNGKVTKSYSFDEIRKNAQLNIELEAAHH

SEQ ID NO: 43 - protein, 327 aa, Synthetic construct
MYRMQLLSCIALSLALVTNSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNKRSKKGRLSLHRTPAGNFVTLEEGKGDLEEYGQDLLHTVFKNGKVTKSYSFDEIRKNAQLNIELEAAHH

SEQ ID NO: 44 - protein, 307 aa, synthetic construct
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNKRSKKGRLSLHRTPAGNFVTLEEGKGDLEEYGQDLLHTVFKNGKVTKSYSFDEIRKNAQLNIELEAAHH

SEQ ID NO: 45 - protein, 327 aa, Synthetic construct
MYRMQLLSCIALSLALVTNSCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNKRSKKGRLSLHRTPAGNFVTLEEGKGDLEEYGQDLLHTVFKNGKVTKSYSFDEIRKNAQLNIELEAAHH

SEQ ID NO: 46 - protein, 327 aa, Synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 47 - protein, 347 aa, Synthetic construct
MYRMQLLSCIALSLALVTNSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 48 - protein, 327 aa, Synthetic construct
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 49 - protein, 347 aa, Synthetic construct
MYRMQLLSCIALSLALVTNSCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 50 - protein, 315 aa, Synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSFKDPVADPNKRSKKGRLSLHRTPAGNFVTLEEGKGDLEEYGQDLLHTVFKNGKVTKSYSFDEIRKNAQLNIELEAAHH

SEQ ID NO: 51 - protein, 335 aa, Synthetic construct
MYRMQLLSCIALSLALVTNSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSFKDPVADPNKRSKKGRLSLHRTPAGNFVTLEEGKGDLEEYGQDLLHTVFKNGKVTKSYSFDEIRKNAQLNIELEAAHH

SEQ ID NO: 52 - protein, 315 aa, Synthetic construct
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSFKDPVADPNKRSKKGRLSLHRTPAGNFVTLEEGKGDLEEYGQDLLHTVFKNGKVTKSYSFDEIRKNAQLNIELEAAHH

SEQ ID NO: 53 - protein, 335 aa, Synthetic construct
MYRMQLLSCIALSLALVTNSCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSFKDPVADPNKRSKKGRLSLHRTPAGNFVTLEEGKGDLEEYGQDLLHTVFKNGKVTKSYSFDEIRKNAQLNIELEAAHH

SEQ ID NO: 54 - protein, 299 aa, Synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLSLHRTPAGNFVTLEEGKGDLEEYGQDLLHTVFKNGKVTKSYSFDEIRKNAQLNIELEAAHH

SEQ ID NO: 55 - protein, 319 aa, Synthetic construct
MYRMQLLSCIALSLALVTNSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLSLHRTPAGNFVTLEEGKGDLEEYGQDLLHTVFKNGKVTKSYSFDEIRKNAQLNIELEAAHH

SEQ ID NO: 56 - protein, 299 aa, Synthetic construct
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLSLHRTPAGNFVTLEEGKGDLEEYGQDLLHTVFKNGKVTKSYSFDEIRKNAQLNIELEAAHH

SEQ ID NO: 57 - protein, 319 aa, Synthetic construct
MYRMQLLSCIALSLALVTNSCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLSLHRTPAGNFVTLEEGKGDLEEYGQDLLHTVFKNGKVTKSYSFDEIRKNAQLNIELEAAHH

SEQ ID NO: 58 - protein, 293 aa, Synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPAGNFVTLEEGKGDLEEYGQDLLHTVFKNGKVTKSYSFDEIRKNAQLNIELEAAHH

SEQ ID NO: 59 - protein, 313 aa, synthetic construct
MYRMQLLSCIALSLALVTNSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPAGNFVTLEEGKGDLEEYGQDLLHTVFKNGKVTKSYSFDEIRKNAQLNIELEAAHH

SEQ ID NO: 60 - protein, 293 aa, Synthetic construct
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPAGNFVTLEEGKGDLEEYGQDLLHTVFKNGKVTKSYSFDEIRKNAQLNIELEAAHH

SEQ ID NO: 61 - protein, 313 aa, Synthetic construct
MYRMQLLSCIALSLALVTNSCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPAGNFVTLEEGKGDLEEYGQDLLHTVFKNGKVTKSYSFDEIRKNAQLNIELEAAHH

SEQ ID NO: 62 - DNA, 60 bp, Synthetic construct
  source 1..60: mol_type = other DNA
atgtacaggatgcagctgctgagctgcatcgccctgagcctggccctggtgaccaacagc

SEQ ID NO: 63 - DNA, 726 bp, Synthetic construct
  source 1..726: mol_type = other DNA
atgtacaggatgcagctgctgagctgcatcgccctgagcctggccctggtgaccaacagctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 64 - DNA, 666 bp, Synthetic construct
  source 1..666: mol_type = other DNA
tgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 65 - DNA, 726 bp, Synthetic construct
  source 1..726: mol_type = other DNA
atgtacaggatgcagctgctgagctgcatcgccctgagcctggccctggtgaccaacagctgccccccctgccccgcccccgaggccgccggcggccccagcgtgttcctgttcccccccaagcccaaggacaccctgtacatcaccagggagcccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 66 - DNA, 666 bp, Synthetic construct
  source 1..666: mol_type = other DNA
tgccccccctgccccgcccccgaggccgccggcggccccagcgtgttcctgttcccccccaagcccaaggacaccctgtacatcaccagggagcccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 67 - DNA, 45 bp, Synthetic construct
  source 1..45: mol_type = other DNA
ggcggcggcggcagcggcggcggcggcagcggcggcggcggcagc

SEQ ID NO: 68 - DNA, 924 bp, Synthetic construct
  source 1..924: mol_type = other DNA
tgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcggcggcagcggcggcggcggcagcggcggcggcggcagcaacaagaggagcaagaagggcaggctgagcctgcacaggacccccgccggcaacttcgtgaccctggaggagggcaagggcgacctggaggagtacggccaggacctgctgcacaccgtgttcaagaacggcaaggtgaccaagagctacagcttcgacgagatcaggaagaacgcccagctgaacatcgagctggaggccgcccaccactga

SEQ ID NO: 69 - DNA, 924 bp, synthetic construct; identical to SEQ ID NO: 68
  source 1..924: mol_type = other DNA

SEQ ID NO: 70 - DNA, 924 bp, Synthetic construct
  source 1..924: mol_type = other DNA
tgccccccctgccccgcccccgaggccgccggcggccccagcgtgttcctgttcccccccaagcccaaggacaccctgtacatcaccagggagcccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcggcggcagcggcggcggcggcagcggcggcggcggcagcaacaagaggagcaagaagggcaggctgagcctgcacaggacccccgccggcaacttcgtgaccctggaggagggcaagggcgacctggaggagtacggccaggacctgctgcacaccgtgttcaagaacggcaaggtgaccaagagctacagcttcgacgagatcaggaagaacgcccagctgaacatcgagctggaggccgcccaccactga

SEQ ID NO: 71 - DNA, 984 bp, Synthetic construct
  source 1..984: mol_type = other DNA
atgtacaggatgcagctgctgagctgcatcgccctgagcctggccctggtgaccaacagctgccccccctgccccgcccccgaggccgccggcggccccagcgtgttcctgttcccccccaagcccaaggacaccctgtacatcaccagggagcccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcggcggcagcggcggcggcggcagcggcggcggcggcagcaacaagaggagcaagaagggcaggctgagcctgcacaggacccccgccggcaacttcgtgaccctggaggagggcaagggcgacctggaggagtacggccaggacctgctgcacaccgtgttcaagaacggcaaggtgaccaagagctacagcttcgacgagatcaggaagaacgcccagctgaacatcgagctggaggccgcccaccactga

SEQ ID NO: 72 - protein, 4 aa, Synthetic construct
GGGS

SEQ ID NO: 73 - protein, 5 aa, Synthetic construct
GGGGS

SEQ ID NO: 74 - protein, 6 aa, Synthetic construct
GSGSGS

(19) United States
(12) Patent Application Publication
(10) Pub. No.: US 2026/0317733 A1
(43) Pub. Date: Oct. 15, 2026

(54) NAMPT FUSION PROTEINS

(71) Applicant: Monash University, Clayton, Victoria (AU)

(72) Inventors: Peter CURRIE, Clayton, Victoria (AU); Mikael MARTINO, Clayton, Victoria (AU)

(21) Appl. No.: 19/481,113

(22) PCT Filed: May 3, 2024
(86) PCT No.: PCT/AU2024/050432

§ 371 Date: Nov. 3, 2025

(30) Foreign Application Priority Data
May 3, 2023 (AU) 2023901320

(51) Int. Cl. C12N 9/10; A61K 38/00; A61P 21/00; C07K 14/55

(52) U.S. Cl. CPC C12N 9/1077; A61P 21/00; C07K 14/55; A61K 38/00; C07K 2319/30; C12Y 204/02012

(57) ABSTRACT

The present invention relates to productive tissue repair and regeneration, and in particular fusion proteins, compositions including said fusion proteins, and methods of using said fusion proteins or compositions for productive tissue repair and regeneration. In one aspect, the invention provides fusion protein comprising: a NAMPT cytokine finger (cif) polypeptide and an Fc region of an antibody.

### FIELD OF THE INVENTION

The present invention relates to productive tissue repair and regeneration, and in particular fusion proteins, compositions including said fusion proteins, and methods of using said fusion proteins or compositions for productive tissue repair and regeneration.

### RELATED APPLICATION

This application claims the benefit of priority from Australian provisional application no. 2023901320 filed 3 May 2023, the entire disclosure of which is incorporated herein by reference.

### BACKGROUND OF THE INVENTION

Skeletal muscle typically forms approximately 40% of a body mass in human adults. It is formed during development by myogenesis wherein paired blocks of paraxial mesoderm known as somites give rise to a transitory myotome that forms muscle stem cells and expands to form an integrated and complex musculature through fusion of myoblasts to the surface of myotubes. In a further stage of myogenesis, muscle stem cells (called satellite cells) migrate to occupy a niche between the sarcolemma and basal lamina of individual myofibers. Amniotes are born with a full set of muscle fibres and, in adults, muscle repair is generally effected through an increase in the size of existing fibres. Throughout life, homeostasis, growth, regeneration and repair of muscle tissue is driven by mesoderm derived skeletal muscle resident stem cells. At a molecular level, quiescent satellite cells require and express the transcription factor PAX7 and also express PAX3. Following skeletal muscle damage, some satellite cells become activated, proliferate to form myoblasts that differentiate and fuse to form new myofibres or merge with and repair damaged muscle fibres. This myogenic programme is governed by myogenic regulatory factors, MYF5, MYOD, MYOG and MRF4. A wealth of other factors and cells associated with the muscle niche are thought to be involved in the complex cellular processes and final production of functional tissue in homeostatic and regenerative contexts. Hence it has been difficult to date to identify the source and nature of the signals that stimulate satellite cell activation and proliferation.

The satellite cell is archetypal of a unipotent tissue-resident stem cell that occupies a specific anatomical niche within a differentiated tissue. Decades of research have revealed the extraordinary capacity of this system to effectively coordinate muscle repair in response to a wide variety of insults. Despite this demonstrated regenerative capacity, transplantation of isolated muscle stem cells has yet to provide therapeutic impact, and pro-regenerative treatments that stimulate muscle stem cells are entirely lacking at this juncture.

There is a need for new and/or improved molecules, compositions and methods for use in myoblast based therapy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, there is provided a fusion protein comprising:
(i) a NAMPT cytokine finger (cif) polypeptide; and
(ii) an Fc region of an antibody.
   Preferably wherein the Fc region has a reduced affinity for an Fc receptor compared to wild-type or naturally occurring Fc regions.

The NAMPT cif polypeptide and Fc region of an antibody may be joined directly or via a linker sequence.

In one embodiment, the NAMPT cif polypeptide is C-terminal to the Fc region of the antibody. Preferably, the N-terminus of the NAMPT cif polypeptide is joined directly or via a linker sequence to the C-terminus of the Fc region of an antibody.

In one embodiment, the NAMPT cif polypeptide is N-terminal to the Fc region of the antibody. Preferably, the C-terminus of the NAMPT cif polypeptide is joined directly or via a linker sequence to the N-terminus of the Fc region of an antibody.

In one embodiment, the fusion protein comprises a first NAMPT cif polypeptide that is N-terminal to the Fc region of the antibody and a second NAMPT cif polypeptide that is C-terminal to the Fc region of the antibody. Preferably, the C-terminus of the first NAMPT cif polypeptide is joined directly or via a linker sequence to the N-terminus of the Fc region of an antibody. Preferably, the N-terminus of the second NAMPT cif polypeptide is joined directly or via a linker sequence to the C-terminus of the Fc region of an antibody.

In one embodiment, the NAMPT cytokine finger (cif) polypeptide comprises, consists essentially of or consists of an amino acid sequence of full length NAMPT cif.

In another embodiment, the NAMPT cytokine finger (cif) polypeptide comprises, consists essentially of or consists of an amino acid sequence of a truncated NAMPT cif.

In another embodiment, the NAMPT cytokine finger (cif) polypeptide comprises, consists essentially of or consists of an amino acid sequence of a fragment of NAMPT cif.

In one embodiment, the NAMPT cytokine finger (cif) polypeptide comprises, consists essentially of or consists of an amino acid sequence of NAMPT₄₀₂₋₄₉₁. Preferably, the numbering corresponds to that of full length NAMPT as shown in SEQ ID NO: 19.

In one embodiment, the NAMPT cytokine finger (cif) polypeptide comprises, consists essentially of or consists of an amino acid sequence of NAMPT₄₁₄₋₄₉₁. Preferably, the numbering corresponds to that of full length NAMPT as shown in SEQ ID NO: 19.

In one embodiment, the NAMPT cytokine finger (cif) polypeptide comprises, consists essentially of or consists of an amino acid sequence of NAMPT₄₂₂₋₄₉₁. Preferably, the numbering corresponds to that of full length NAMPT as shown in SEQ ID NO: 19.

In one embodiment, the NAMPT cytokine finger (cif) polypeptide comprises, consists essentially of or consists of an amino acid sequence of NAMPT₄₃₀₋₄₉₁.

Preferably, the numbering corresponds to that of full length NAMPT as shown in SEQ ID NO: 19.

In one embodiment, the NAMPT cytokine finger (cif) polypeptide comprises, consists essentially of or consists of an amino acid sequence of NAMPT₄₃₆₋₄₉₁. Preferably, the numbering corresponds to that of full length NAMPT as shown in SEQ ID NO: 19.

In one embodiment, the fusion protein may further comprise a signal sequence, for example an IL-2 signal sequence. In one embodiment, the signal sequence comprises, consists essentially of or consists of the amino acid sequence as shown in SEQ ID NO: 36.

In any embodiment, the NAMPT cif polypeptide comprises, consists essentially of or consists of the amino acid sequence set out in any one of SEQ ID Nos: 1, 2 or 3. Preferably, the NAMPT cif polypeptide comprises, consists essentially of or consists of a truncated amino acid sequence of SEQ ID NO: 1.

In any embodiment, the truncated NAMPT cif may have an N-terminal truncation and/or a C-terminal truncation compared to full length NAMPT cif. In one embodiment, the N- and/or C-terminal truncation is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids. Preferably, the truncation is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids N- and/or C-terminal of the amino acid sequence of, or equivalent to, SEQ ID NO: 1.

In any embodiment, the truncation is of the N-terminal residues 1-12, 1-20, 1-28 or 1-35 of cif. Preferably, the truncation is of, or equivalent to, the N-terminal residues 1-12, 1-20, 1-28 or 1-35 of the amino acid sequence set forth in SEQ ID NO: 1.

In one embodiment, the NAMPT cif polypeptide comprises, consists essentially of or consists of an amino acid sequence of any one of SEQ ID Nos: 1, 2, 3, 4, 5, 6, 7, 8, 9, 10 or 11, or an amino acid sequence that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 1, 2, 3, 4, 5, 6, 7, 8, 9, 10 or 11. Alternatively, the NAMPT cif polypeptide comprises, consists essentially of or consists of an amino acid sequence of any one of SEQ ID Nos: 1, 2, 3, 4, 5, 6, 7, 8, 9, 10 or 11, or an amino acid sequence that is equal to, or at least, 70%, 75%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID Nos: 1, 2, 3, 4, 5, 6, 7, 8, 9, 10 or 11.

In one embodiment, % identity or identical to a sequence means that the polypeptide has the same length, for example number of amino acids, but the amino acids across that length are only 70%, 75%, 80%, 85%, 86%, 87%, 88%, 89%, 90%, 91%, 92%, 93%, 94%, 95%, 96%, 97%, 98%, 99%, or 100% identical. Typically, the only differences in amino acid identity is a result of conservative substitutions (for example those outlined in Table 3 below).

In one embodiment, the NAMPT cif polypeptide is equal to, or less than, about 110, about 109, about 108, about 107, about 106, about 105, about 104, about 103, about 102, about 101, about 100, about 99, about 98, about 97, about 96, about 95, about 94, about 93, about 92, about 91, about 90, about 89, about 88, about 87, about 86, about 85, about 84, about 83, about 82, about 81, about 80, about 79, about 78, about 77, about 76, about 75, about 74, about 73, about 72, about 71, about 70, about 69, about 68, about 67, about 66, about 65, about 64, about 63, about 62, about 61, about 60, about 59, about 58, about 57, or about 56 amino acids in length.

In one embodiment, the NAMPT cif polypeptide is equal to, or less than, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, or 56 amino acids in length.

In one embodiment, the amino acid sequence of the NAMPT cif polypeptide that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 1, 2, 3, 4, 5, 6, 7, 8, 9, 10 or 11 is equal to, or less than, about 110, about 109, about 108, about 107, about 106, about 105, about 104, about 103, about 102, about 101, about 100, about 99, about 98, about 97, about 96, about 95, about 94, about 93, about 92, about 91, about 90, about 89, about 88, about 87, about 86, about 85, about 84, about 83, about 82, about 81, about 80, about 79, about 78, about 77, about 76, about 75, about 74, about 73, about 72, about 71, about 70, about 69, about 68, about 67, about 66, about 65, about 64, about 63, about 62, about 61, about 60, about 59, about 58, about 57, or about 56 amino acids in length.

In one embodiment, the amino acid sequence of the NAMPT cif polypeptide that is equal to, or at least, 70%, 75%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID Nos: 1, 2, 3, 4, 5, 6, 7, 8, 9, 10 or 11 is equal to, or less than, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, or 56 amino acids in length.

In any embodiment, the NAMPT cif polypeptide comprises an amino acid sequence having 1, 2, 3, 4, 5, 6, 7 or 8 conservative (for example those outlined in Table 3 below) or non-conservative amino acid substitutions, deletions or additions to the above sequences, and retains CCR5 or tissue stem cell interacting activity. Preferably the conservative or non-conservative amino acid substitutions, deletions or additions are not of the amino acids 431 to 435 or 472 to 491 (numbering corresponding to human NAMPT, e.g. SEQ ID NO: 19).

In one embodiment, the NAMPT cif polypeptide comprises, consists essentially of or consists of the amino acid sequence of SEQ ID NO: 8 with an N-terminal truncation. Preferably the N-terminal truncation is less than 6 amino acids.

In one embodiment, any polypeptide as described herein comprises the C-terminal alpha helix present in the cif motif.

In one embodiment, the NAMPT cif polypeptide consists of an amino acid sequence having equal to, or at least, 85% sequence identity to the amino acid sequence of SEQ ID NO: 1. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 1.

In one embodiment, the NAMPT cif polypeptide consists of an amino acid sequence having equal to, or at least, 85% sequence identity to the amino acid sequence of SEQ ID NO: 4 or 5. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 4 or 5.

In one embodiment, the NAMPT cif polypeptide consists of an amino acid sequence having equal to, or at least, 85% sequence identity to the amino acid sequence of SEQ ID NO: 6 or 7. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 6 or 7.

In one embodiment, the NAMPT cif polypeptide consists of an amino acid sequence having equal to, or at least, 85% sequence identity to the amino acid sequence of SEQ ID NO: 8 or 9. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 8 or 9.

In one embodiment, the NAMPT cif polypeptide consists of an amino acid sequence having equal to, or at least, 85% sequence identity to the amino acid sequence of SEQ ID NO: 10 or 11. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 10 or 11.

In one embodiment, the truncated NAMPT cif has an N terminal truncation of, or equivalent to, residues 402 to 413 of SEQ ID NO: 19, and wherein the polypeptide has at least 85% sequence identity to the amino acid sequence of 414 to 491 of SEQ ID NO: 19. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 414 to 491 of SEQ ID NO: 19.

In one embodiment, the truncated NAMPT cif has an N terminal truncation of, or equivalent to, residues 402 to 421 of SEQ ID NO: 19, and wherein the polypeptide has at least 85% sequence identity to the amino acid sequence of 422 to 491 of SEQ ID NO: 19. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 422 to 491 of SEQ ID NO: 19.

In one embodiment, the truncated NAMPT cif has an N terminal truncation of, or equivalent to, residues 402 to 429 of SEQ ID NO: 19, and wherein the polypeptide has at least 85% sequence identity to the amino acid sequence of 430 to 491 of SEQ ID NO: 19. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 430 to 491 of SEQ ID NO: 19.

In one embodiment, the truncated NAMPT cif has an N terminal truncation of, or equivalent to, residues 402 to 435 of SEQ ID NO: 19, and wherein the polypeptide has at least 85% sequence identity to the amino acid sequence of 436 to 491 of SEQ ID NO: 19. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 436 to 491 of SEQ ID NO: 19.

In one embodiment, the fusion protein may be in monomeric, dimeric or multimeric form. To facilitate dimerization any polypeptide described herein may be modified to allow homo or heterodimerisation. The modification may be the addition of an amino acid, either natural or non-natural, that forms a covalent bond, for example as cysteine that forms a disulphide bond. Typically, the addition of an amino acid, such as a cysteine, that forms a covalent bond is at the N- or C-terminus of the polypeptide. In one embodiment, the polypeptide comprises, consists essentially of or consists of an amino acid sequence of any one of SEQ ID Nos: 1, 3, 4, 5, 6, 7, 8, 9, 10 or 11 with an additional cysteine at the N- or C-terminus.

In another embodiment, there is provided a dimeric fusion protein formed by covalent bonded, preferably disulphide bonded, monomers of a polypeptide described herein.

In another embodiment, the fusion protein comprises, consists essentially of or consists of 2 or more full length NAMPT cif molecules, truncated NAMPT cif molecules, or fragments thereof as described herein.

The Fc region of an antibody may be an Fc region of an IgG, IgA, IgD, IgE, or IgM. Preferably the Fc region is from an IgG antibody, such as an IgG1, an IgG2, an IgG2b, an IgG3 or an IgG4 antibody.

Preferably, the Fc region of the fusion protein comprises two heavy chain fragments, more preferably the CH2 and CH3 domains of said heavy chain.

In any embodiment, the Fc region of the fusion protein comprises a hinge region. The hinge region may be derived from an immunoglobulin-like molecule. For example, the hinge region may comprise or consist of a hinge region from an IgG1, IgG2, IgG3 or IgG4, including a fragment thereof.

In any embodiment, the Fc region of an antibody may not comprise one or more of a VH, VL, light chain CH1 and heavy chain CH1.

In any embodiment, the Fc region of an antibody comprises, consists essentially of or consists of a hinge region, CH2 and CH3 domain.

In any embodiment, the fusion protein may comprise a linker between the NAMPT cif polypeptide and the Fc region of the antibody, such as between the C-terminus of the Fc region and the N-terminus of the NAMPT cif polypeptide.

The Fc region preferably comprises one or more amino acid substitutions for reducing affinity for the Fc receptor (FcR, including any of FcγRI, FcγRII and FcγRIII) and thereby reducing the ability of the fusion protein to elicit antibody-dependent cell-mediated toxicity (ADCC). Such substitutions are well known in the art and include, but are not limited to the “DANA”, and “LALA” amino acid substitutions and variations thereof, as further defined herein.

In preferred embodiments, the fusion protein has an affinity for FcR that is less than about 250 nM, preferably less than 500 nM, less than 1000 nM, most preferably less than 2000 nM.

In another embodiment, the Fc region of an antibody may include one or more amino acid substitutions for increasing the serum half-life of the Fc and/or the fusion protein.

In one embodiment the amino acid substitutions for increasing the serum half-life of the Fc and/or the fusion protein is a substitution at one or more of M252, S254 and T256. Preferably, the substitutions are one or more of M252Y, S254T and T256E.

In one embodiment, the Fc region of an antibody comprises, consists essentially of or consists of an amino acid sequence as shown in SEQ ID NOs: 38 or 40.

In any embodiments, the fusion protein may comprise a linker between the NAMPT cif polypeptide and the Fc region of the antibody, such as between the N-terminus of the Fc regions and the C-terminus of the NAMPT polypeptide.

Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids.

In any embodiment, the linker is a peptide linker. In any embodiment, the peptide linker links a NAMPT polypeptide with an Fc region of an antibody. In any embodiment, the peptide linker can include the amino acid sequence Gly-Gly-Ser (GGS), Gly-Gly-Gly-Ser (GGGS; SEQ ID NO: 72) or Gly-Gly-Gly-Gly-Ser (GGGGS; SEQ ID NO: 73). In some aspects, the peptide linker can include the amino acid sequence GGGGS (a linker of 6 amino acids in length) or even longer. The linker may a series of repeating glycine and serine residues (GS) of different lengths, i.e., (GS) n where n is any number from 1 to 15 or more. For example, the linker may be (GS) 3 (i.e., GSGSGS; SEQ ID NO: 74) or longer (GS) 11 or longer. It will be appreciated that n can be any number including 1, 2, 3, 4, 5, 6, 7, 8, 9, 10, 11 or more. Fusion proteins having linkers of such length are included within the scope of the present invention. Preferably n is no more than 3 (ie such that when n equals 3 the linker is GSGSGS; SEQ ID NO: 74). In a preferred embodiment the linker comprises or consists of the sequence shown in SEQ ID NO: 41.

In one embodiment, the fusion protein comprises an amino acid sequence as shown in any one of SEQ ID NOs: 42 to 61.

In any embodiment, a fusion protein as described herein stimulates muscle stem cell (e.g. satellite) proliferation to a level that is equal to or greater than full length NAMPT, for example comprising, consisting essentially of or consisting of SEQ ID NO: 19, or equal to or greater than full length NAMPTcif without an Fc region of an antibody, for example comprising, consisting essentially of or consisting of SEQ ID NO: 1 or 2. Preferably, muscle stem cell (e.g. satellite) proliferation is determined by an assay described herein, including in Example 1.

In any embodiment, a fusion protein described herein stimulates TLR4 activation to a significantly lower level than full length NAMPT. For example, stimulates TLR4 activation to a level equal to, or less than, 90%, 85%, 80%, 75%, 70%, 65%, 60%, 55%, 50%, 45%, 40%, 35%, 30%, 25%, 20%, 15% or 10% full length NAMPT (for example, a polypeptide comprising or consisting of an amino acid sequence of SEQ ID NO: 19). In any embodiment, a polypeptide or fusion protein stimulates TLR4 activation to the same, or not-significantly different, level as the cytokine finger motif of NAMPT without an Fc region of an antibody (for example, a polypeptide consisting of the amino acid sequence of SEQ ID NO: 1).

In another embodiment, the fusion protein is a dimer wherein each monomer comprises:
(i) a NAMPT cytokine finger (cif) polypeptide; and
(ii) an Fc region of an antibody.

Preferably, each monomer in the dimer comprises, consists essentially of or consists of a hinge region, CH2, CH3 and NAMPT cif polypeptide. Typically, each monomer has the following arrangement, from N-terminus to C-terminus: a hinge region, CH2, CH3 and NAMPT cif polypeptide.

In one embodiment, the monomer comprises, consists essentially of or consist of an amino acid sequence as shown in any one of SEQ ID NOs: 42 to 61.

In another aspect, the present invention also provides isolated nucleic acids encoding a fusion protein as described herein, for example, comprising, consisting essentially of or consisting of the amino acid sequence of any one of SEQ ID Nos: 42 to 61. In one embodiment, a nucleic acid molecule encoding a fusion protein described herein comprises, consists essentially of or consists of the polynucleotide sequence set out in any one of SEQ ID NO: 68 to 71, or a polynucleotide sequence that has at least 70%, 75%, 80%, 85%, 86%, 87%, 88%, 89%, 90%, 91%, 92%, 93%, 94%, 95%, 96%, 97%, 98%, 99%, or 100% sequence identity thereto.

The nucleic acid molecule may be an RNA or DNA or RNA:DNA or a chemically modified form thereof. For example, the nucleic acid may be in the form of a viral or non-viral vector.

In another aspect, the present invention provides vectors comprising said nucleic acids, optionally, operably linked to control sequences.

In another aspect, the present invention provides host cells containing the vectors, and methods for producing and optionally recovering the fusion proteins.

In another aspect, the present invention provides a cell expressing a fusion protein as described herein.

In another aspect, the present invention provides compositions providing chemokine receptor interaction or binding activity or muscle tissue stem cell interacting activity for use in stimulating muscle regeneration. In one embodiment, the present application provides compositions providing chemokine receptor interaction or binding activity or satellite cell binding or interacting activity for use in stimulating muscle regeneration without fibrosis or substantially without fibrosis. In one embodiment, the chemokine receptor is a CCR5 chemokine receptor or a tissue stem cell receptor that binds NAMPT, including a tissue stem cell receptor that binds NAMPTcif. In one embodiment the composition, comprising a cell or other agent that provides CCR5 interacting activity, binds to tissue stem cells, particularly muscle stem cells.

In one embodiment, the composition comprises a fusion protein, nucleic acid, vector, or cell as described herein and a pharmaceutically acceptable carrier, diluent or excipient. In one embodiment, the composition may further comprise one, two or all of (a) a tissue stem cell (such as a satellite cell) or precursor therefore or progeny thereof, (b) a macrophage or a precursor therefore or progeny thereof, and (c) a scaffold or retentive material.

In one non-limiting embodiment, promoting muscle stem cell chemokine receptor signalling is particularly useful in treating subjects with a muscle injury including volumetric muscle loss injuries or muscle degeneration/atrophy, or muscular or neuromuscular impairments, muscular or neuromuscular degenerative conditions, myopathy, or the propensity therefore. In one embodiment, chemokine receptor binding activity is provided in the form of a cell such as a macrophage or stem cell expressing a fusion protein as described herein.

In one embodiment, the chemokine receptor is a CCR5 receptor.

In one embodiment, the CCR5 receptor is a tissue stem cell or tissue stem cell progeny CCR5 receptor. In one embodiment the CCR5 receptor is a satellite cell or satellite cell progeny CCR5 receptor.

In one embodiment, in vitro, in vivo and ex vivo applications are contemplated.

In another aspect, the present invention provides a method of stimulating proliferation of a stem cell, such as satellite cell proliferation, the method comprising administering to a cell or subject an effective amount of a fusion protein, nucleic acid, vector, composition or cell as described herein, thereby stimulating proliferation of a stem cell.

In another aspect, the present invention provides use of a fusion protein, nucleic acid, vector, composition or cell as described herein as described herein in the manufacture of a medicament for stimulating proliferation of a stem cell, such as satellite cell proliferation.

In another aspect, the present invention provides a fusion protein, nucleic acid, vector, composition or cell as described herein as described herein for use in stimulating proliferation of a stem cell, comprising administering to a cell or subject an effective amount of the fusion protein, nucleic acid, vector, composition or cell, thereby stimulating proliferation of a stem cell.

In another aspect, the present invention provides a method of stimulating muscle tissue regeneration in a subject, the method comprising administering to a muscle of the subject an effective amount of a fusion protein, nucleic acid, vector, composition or cell as described herein, thereby stimulating muscle tissue regeneration.

In another aspect, the present invention provides use of a fusion protein, nucleic acid, vector, composition or cell as described herein as described herein in the manufacture of a medicament for stimulating muscle tissue regeneration in a subject.

In another aspect, the present invention provides a fusion protein, nucleic acid, vector, composition or cell as described herein as described herein for use in stimulating muscle tissue regeneration in a subject, comprising administering to the subject an effective amount of the fusion protein, nucleic acid, vector, composition or cell, thereby stimulating muscle tissue regeneration.

In any embodiment, the fusion protein is a CCR5 agonist. That is, it stimulates receptor signalling or downstream events such as satellite cell activation and proliferation. In one embodiment, the fusion protein specifically activates tissue stem cells. In one embodiment, the fusion protein specifically activates satellite cells.

As described herein, in one embodiment, tissue regeneration stimulated by the method is associated with minimal fibrosis. Thus, in another aspect, the present application provides fusion proteins, compositions, cells and methods for reducing fibrosis development in a patient or biological tissue subject to regenerative treatment.

In one embodiment, the present application provides a method suitable for regenerating muscle tissue in vitro, in vivo or ex vivo. Accordingly, fusion protein, nucleic acid, vector, composition or cell described herein is proposed for use, or for use in, stem cell based therapies and tissue engineering, including for use in the manufacture of medicaments thereto. In another embodiment fusion protein, nucleic acid, vector, composition or cell described herein is for use, or for use in, artificial meat production in vitro.

In another aspect, the present invention provides a method of stimulating muscle tissue regeneration, the method comprising administering to a muscle an effective amount of a fusion protein, nucleic acid, vector, composition or cell as described herein, wherein the fusion protein, nucleic acid, vector, composition or cell as described herein binds to satellite cells and stimulates satellite cell activation, myoblast proliferation and muscle regeneration and the absence of substantial fibrosis (scar formation).

In another aspect, the present invention provides use of a fusion protein, nucleic acid, vector, composition or cell as described herein as described herein in the manufacture of a medicament for stimulating muscle tissue regeneration, wherein the fusion protein, nucleic acid, vector, composition or cell as described herein binds to satellite cells and stimulates satellite cell activation, myoblast proliferation and muscle regeneration and the absence of substantial fibrosis (scar formation).

In another aspect, the present invention provides a fusion protein, nucleic acid, vector, composition or cell as described herein as described herein for use in stimulating muscle tissue regeneration, comprising administering to the cell or subject an effective amount of the fusion protein, nucleic acid, vector, composition or cell, thereby stimulating muscle tissue regeneration, wherein the fusion protein, nucleic acid, vector, composition or cell binds to satellite cells and stimulates satellite cell activation, myoblast proliferation and muscle regeneration and the absence of substantial fibrosis (scar formation).

In another aspect, the present invention provides a method of stimulating muscle tissue regeneration in a subject where inflammation is undesirable, the method comprising administering to a muscle an effective amount of a fusion protein, nucleic acid, vector, composition or cell as described herein, thereby stimulating muscle tissue regeneration in this subject.

In another aspect, the present invention provides use of a fusion protein, nucleic acid, vector, composition or cell as described herein in the manufacture of a medicament for stimulating muscle tissue regeneration in a subject where inflammation is undesirable.

In another aspect, the present invention provides a fusion protein, nucleic acid, vector, composition or cell as described herein for use in stimulating muscle tissue regeneration in a subject where inflammation is undesirable, comprising administering to a muscle an effective amount of the fusion protein, nucleic acid, vector, composition or cell as described herein, thereby stimulating muscle tissue regeneration in this subject.

In any embodiment, the inflammation that is undesirable is inflammation mediated by TLR activation, preferably TLR4 activation.

In any embodiment, the subject may be diagnosed with an inflammatory myopathy. Exemplary inflammatory myopathies include polymyositis, dermatomyositis, inclusion body myositis, necrotizing autoimmune myopathy.

In this aspect, the present invention provides a method of treating an inflammatory myopathy in a subject, the method comprising administering to a muscle of the subject an effective amount of a fusion protein, nucleic acid, vector, composition or cell as described herein, thereby treating an inflammatory myopathy. Preferably the inflammatory myopathy is polymyositis, dermatomyositis, inclusion body myositis or necrotizing autoimmune myopathy.

In another aspect, the present invention provides use of a fusion protein, nucleic acid, vector, composition or cell as described herein in the manufacture of a medicament for treating an inflammatory myopathy in a subject. Preferably the inflammatory myopathy is polymyositis, dermatomyositis, inclusion body myositis or necrotizing autoimmune myopathy.

In another aspect, the present invention provides a fusion protein, nucleic acid, vector, composition or cell as described herein for use in treating an inflammatory myopathy in a subject, comprising administering to a muscle of the subject an effective amount of the fusion protein, composition or cell as described herein, thereby treating an inflammatory myopathy. Preferably the inflammatory myopathy is polymyositis, dermatomyositis, inclusion body myositis or necrotizing autoimmune myopathy.

Reference to NAMPT and CCR5 includes homologues and orthologs thereof include from any animal including mammals, non-mammalian vertebrates, fish and birds.

In another aspect, the present application provides a method of stimulating muscle tissue regeneration, the method comprising administering to a muscle an effective amount of a composition comprising a cell comprising or encoding a fusion protein as described herein, and optionally a component that enhances delivery to or retention in the muscle, wherein the fusion protein as described herein binds to satellite cells and stimulates myoblast proliferation and muscle regeneration.

In another aspect, the present application provides use of a composition comprising a cell comprising or encoding a fusion protein as described herein, and optionally a component that enhances delivery to or retention in the muscle, in the manufacture of medicament for stimulating muscle tissue regeneration, wherein the fusion protein as described herein binds to satellite cells and stimulates myoblast proliferation and muscle regeneration.

In another aspect, the present application provides a composition comprising a cell comprising or encoding a fusion protein as described herein, and optionally a component that enhances delivery to or retention in the muscle for use in stimulating muscle tissue regeneration, comprising administering to a muscle an effective amount of a composition comprising a cell comprising or encoding a fusion protein as described herein, and optionally a component that enhances delivery to or retention in the muscle, wherein the fusion protein as described herein binds to satellite cells and stimulates myoblast proliferation and muscle regeneration.

In any embodiment, the cell is a macrophage. In one embodiment the macrophage is isolated from tissue. In one embodiment, the macrophage is induced from stem cells such as bone marrow precursors or iPSC. In one embodiment, the macrophage or macrophage precursor (a monocyte) is isolated from a supply tissue such as, but not limited to blood, lymph, bone marrow) and then subjected to in vitro cell or tissue culture to induce the desired tissue niche directed phenotype. In one embodiment, the cell composition is cryopreserved and/or contains a delivery agent.

As known in the art, macrophages may be generated in vitro from stem cells by various means. Macrophages generated from stem cells, such as BMSC, in the presence of IFNγ or LPS are generally considered as “inflammatory” macrophages referred to as “M1 macrophages.” Those generated in the presence of IL-4 or IL-10 have what is called a “pro-resolution” activity and are referred to as “M2” macrophages.

In any embodiment, the subject macrophage expresses M2 macrophage markers.

In any embodiment, the macrophage cell expresses one or two or three or four or five of mmp9, arg2, mmp13a, L-plastin and cd163.

In any embodiment, the macrophage cell expresses one or two or three or four or five of MRC1, CD200R, CD209, ARG1 and CHI3L3.

In any embodiment, the macrophage subset expresses prox1a and pou2f3.

In any embodiment, the composition further comprises a stem cell and/or a macrophage cell.

In any embodiment, the stem cell is a satellite cell. In another embodiment the stem cell is a unipotent or multipotent stem cell.

In any embodiment of the method, the active ingredient or main ingredient is, or is only, a fusion protein described herein.

In any embodiment, the fusion protein described herein further comprises one or more moieties such as a linker, stability enhancing, signalling enhancing, delivery enhancing or label moiety.

In another aspect, the present invention provides compositions comprising a fusion protein, nucleic acid, vector, or cell as defined herein. Pharmaceutical and physiologically active compositions are provided. Cellular compositions are expressly provided.

In one embodiment, the cell is a macrophage. In one embodiment the macrophage is isolated from tissue. In one embodiment, the macrophage is induced from stem cells such as bone marrow precursors or iPSC. In one embodiment, the macrophage or macrophage precursor (a monocyte) is isolated from a supply tissue such as, but not limited to blood, lymph, bone marrow) and then subjected to in vitro cell or tissue culture to induce the desired tissue niche directed phenotype. In one embodiment, the cell composition is cryopreserved and/or contains a delivery agent.

As known in the art, macrophages may be generated in vitro from stem cells by various means. Macrophages generated from stem cells, such as BMSC, in the presence of IFNg or LPS are generally considered as “inflammatory” macrophages referred to as “M1 macrophages.” Those generated in the presence of IL-4 or IL-10 have what is called a “pro-resolution” activity and are referred to as “M2” macrophages.

In one embodiment, the subject macrophage expresses M2 macrophage markers.

In one embodiment, the macrophage cell expresses one or two or three or four or five of mmp9, arg2, mmp13a, L-plastin and cd163.

In any embodiment, the macrophage cell expresses one or two or three or four or five of MRC1, CD200R, CD209, Arg1 and Chi313.

In one other embodiment, the macrophage subset expresses prox1a and pou2f3.

In one embodiment, the composition comprises or is administered together with a supporting material such as a hydrogel, glue, foam or retentive material, scaffold etc. Delicate structures are generally suitable for enabling more delicate tissue regeneration. As examples, materials can be used which are quite rapidly absorbed, such as certain fibrin, collagen, hydrogel and alginate formulations. Alternatively, slowly absorbable synthetics can be used, such as poly-4-hydroxybutarate. Silk fibres or even substantially smooth products derived from mammalian origin such as muscle extracellular matrix are also contemplated. Non-absorbable synthetics, such as polypropylene and polyethylene, provide support and reliability. In one embodiment the composition comprises a fibrin hydrogel. In another embodiment, RAFT-acrylamide based support surfaces are provided to enhance tissue regeneration and bioavailability of a fusion protein, nucleic acid, vector, or cell to the target site.

In one embodiment there is provided a composition comprising a fusion protein, nucleic acid, vector, or cell as described herein and any one or two or three or four of: (i) a satellite cell or precursor therefore or progeny thereof (ii) a macrophage or a precursor therefore or progeny thereof (iii) a scaffold (semi-solid or solid support) or retentive material (iv) a tissue delivery enhancing or cell retention moiety.

In one embodiment the scaffold or retentive material is a hydrogel, such as a fibrin or acrylamide hydrogel. In one embodiment, the tissue delivery enhancing or cell retention moiety is an ECM-binding moiety.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Exemplary structure of an Fc-NAMPT₄₂₂₋₄₉₁ fusion protein as encompassed by the present invention. From N- to C-terminus, the structure comprises an IL-2 signalling sequence, a hinge region, an IgG1 Fc domain (with L234A, L235A, M252Y, S254T, T256E substitutions) and a NAMPTcif₄₂₂₋₄₉₁ polypeptide linked to the C-terminus of the IgG1 Fc domain via a (G₄S)₃ linker.

FIG. 2. Human NAMPT variants stimulate human satellite cell proliferation. Human primary satellite cells were treated with 20 nM full length NAMPT (NAMPT), NAMPT₄₂₂₋₄₉₁, IgG1 or IgG1-NAMPT₄₂₂₋₄₉₁ for 48 hours. The media contained the growth factors IGF-1 and FGF-2 as well. Quantification of cell proliferation was performed using CyQuant Proliferation Assay kit. Data are displayed as percentage increase vs. PBS-treated negative control. Treatment with 20% FBS was used as a positive control. Box and whiskers denote median with minimum and maximum values. n=6 independent experiments per protein. Statistical significance is indicated as p-value and determined by one-sample t-test.

FIG. 3. Human NAMPT variants stimulate human endothelial cell proliferation. Human primary endothelial cells derived from umbilical vein were treated with 20 nM full length NAMPT (NAMPT), NAMPT₄₂₂₋₄₉₁, IgG1 or IgG1-NAMPT₄₂₂₋₄₉₁ for 48 hours. Quantification of cell proliferation was performed using CyQuant Proliferation Assay kit. Data are displayed as percentage increase vs. PBS-treated negative control. Treatment with 10% FBS was used as a positive control. Box and whiskers denote median with minimum and maximum values. n=6 independent experiments per protein. Statistical significance is indicated as p-value and determined by one-sample t-test.

FIG. 4. Minimal versions of NAMPT protein enhance proliferation in response to muscle injury in zebrafish larvae. Treatment with IgG1-NAMPT₄₂₂₋₄₉₁ following needle-stick muscle injury induces a significant increase in cell proliferation within the injury zone as compared to 0.1% BSA negative control. Representative images are shown in panel (A). Violin plots in (B) show number of EdU-positive cells (white) in the ‘injury’ zone and ‘external’ represent the two-adjacent somites encompassing the injury zone, (from left to right: n=30 control, 0.1% BSA; n=22 hrNAMPT-treated; n=30 NAMPT₄₂₂₋₄₉₁, n=19 IgG1; n=18 IgG1-NAMPT₄₂₂₋₄₉₁). The thick black lines and dashed black lines within the violin plot indicate the median and quartiles, respectively. Two-way ANOVA with Tuckey's multiple comparison test.

FIG. 5. Fc-NAMPT₄₂₂₋₄₉₁ stimulates proliferation in zebrafish and retains wound site specificity. (A) The effect on wound site (dotted line; skeletal muscle visualized by phalloidin staining) proliferation (EdU, white) was assayed following Fc and Fc-NAMPT₄₂₂₋₄₉₁ treatment in the TU zebrafish line. An individual example is provided for each group and these images are representative of the quantification presented in b. Scale bar, 50 μm. (B) Supplementation with 57 nM Fc fusion Fc-NAMPT₄₂₂₋₄₉₁ increased proliferation versus IgG1 (p<0.0001) by activating muscle stem proliferation (n=55 for Fc, n=48 for Fc-NAMPT₄₂₂₋₄₉₁). Two-way ANOVA with Sidak's multiple comparison test. Exact p-values are indicated.

FIG. 6. Fusion of IgG1 Fc to NAMPT₄₂₂₋₄₉₁ does not interfere with the muscle restoration ability of NAMPT₄₂₂₋₄₉₁. Volumetric muscle loss defect (3×5 mm) was performed on hind limb rectus femoris muscle of 10-week-old C57Bl/6 mice and treated with equimolar variants of NAMPT (0.5 μg; top right panel), NAMPT₄₂₂₋₄₉₁ (0.096 μg; bottom left panel), IgG1Fc NAMPT₄₂₂₋₄₉₁ (0.32 μg; bottom middle panel) or fibrin only (top middle panel) and IgG1-Fc (0.25 μg; bottom right panel) only as controls. (A) Muscle regeneration evaluated at 10 days post injury by microCT expressed as percentage volume restored against contralateral healthy leg as reference (n=11 defects per group). Data are mean±SEM using. One-way ANOVA test with a Benjamin and Hochberg false rate discovery (FDR) was applied to compare all groups with a statistical significance threshold of 0.05. (B) Representative histology of muscle stained with Mason's Trichrome showing longitudinal section of defect. Fibrin (top middle panel), NAMPT (top right panel), NAMPT₄₂₂₋₄₉₁ (bottom left panel), IgG1Fc NAMPT₄₂₂₋₄₉₁ (bottom middle panel), and IgG1-Fc (bottom right panel). Muscle tissue appears as darker grey while fibrotic-like tissue appears as lighter grey. Scale bar, 1 mm.

### SUMMARY OF SEQUENCE LISTING

TABLE 1
Sequences of the invention.

| SEQ ID NO: | DESCRIPTION | SEQUENCE |
|---|---|---|
| SEQ ID NO: 1 | Illustrative Human | SYVVTNGLGINVFKDPVADPNKRSKKGRLSLHRTPA |
| | cytokine finger | GNFVTLEEGKGDLEEYGQDLLHTVFKNGKVTKSYSF |
| | (NAMPT₄₀₂₋₄₉₁, | DEIRKNAQLNIELEAAHH |
| | numbering corresponds | |
| | to full length NAMPT, | |
| | e.g SEQ ID NO: 19) | |
| SEQ ID NO: 2 | Human NAMPT cif | CSYVVTNGLGINVFKDPVADPNKRSKKGRLSLHRTP |
| | monomer as base for | AGNFVTLEEGKGDLEEYGQDLLHTVFKNGKVTKSYS |
| | dimeric or multimeric | FDEIRKNAQLNIELEAAHH |
| | form, with, in this | |
| | embodiment, an n- | |
| | terminal cysteine to | |
| | facilitate dimer formation | |
| SEQ ID NO: 3 | Mouse C-terminal | SYVVTNGLGVNVFKDPVADPNKRSKKGRLSLHRTPA |
| | fragment of NAMPT as | GNFVTLEEGKGDLEEYGHDLLHTVFKNGKVTKSYSF |
| | CCR5 interacting | DEVRKNAQLNIEQDVAPH |
| | peptide | |
| SEQ ID NO: 4 | Truncated hNAMPTcif- | FKDPVADPNKRSKKGRLSLHRTPAGNFVTLEEGKGD |
| | T1 (NAMPT₄₁₄₋₄₉₁, | LEEYGQDLLHTVFKNGKVTKSYSFDEIRKNAQLNIEL |
| | numbering corresponds | EAAHH |
| | to full length NAMPT, | |
| | e.g SEQ ID NO: 19) | |
| SEQ ID NO: 5 | Truncated hNAMPTcif- | HHHHHHHHENLYFQGFKDPVADPNKRSKKGRLSLH |
| | T1 (NAMPT₄₁₄₋₄₉₁, | RTPAGNFVTLEEGKGDLEEYGQDLLHTVFKNGKVTK |
| | numbering corresponds | SYSFDEIRKNAQLNIELEAAHH |
| | to full length NAMPT, | |
| | e.g SEQ ID NO: 19) | |
| | (including HIS tag) | |
| SEQ ID NO: 6 | Truncated hNAMPTcif- | NKRSKKGRLSLHRTPAGNFVTLEEGKGDLEEYGQDL |
| | T2 (NAMPT₄₂₂₋₄₉₁, | LHTVFKNGKVTKSYSFDEIRKNAQLNIELEAAHH |
| | numbering corresponds | |
| | to full length NAMPT, | |
| | e.g SEQ ID NO: 19) | |
| SEQ ID NO: 7 | Truncated hNAMPTcif- | HHHHHHHHENLYFQGNKRSKKGRLSLHRTPAGNFV |
| | T2 (NAMPT₄₂₂₋₄₉₁, | TLEEGKGDLEEYGQDLLHTVFKNGKVTKSYSFDEIR |
| | numbering corresponds | KNAQLNIELEAAHH |
| | to full length NAMPT, | |
| | e.g SEQ ID NO: 19) | |
| | (including HIS tag) | |
| SEQ ID NO: 8 | Truncated hNAMPTcif- | LSLHRTPAGNFVTLEEGKGDLEEYGQDLLHTVFKNG |
| | T3 (NAMPT₄₃₀₋₄₉₁, | KVTKSYSFDEIRKNAQLNIELEAAHH |
| | numbering corresponds | |
| | to full length NAMPT, | |
| | e.g SEQ ID NO: 19) | |
| SEQ ID NO: 9 | Truncated hNAMPTcif- | HHHHHHHHENLYFQGLSLHRTPAGNFVTLEEGKGD |
| | T3 (NAMPT₄₃₀₋₄₉₁, | LEEYGQDLLHTVFKNGKVTKSYSFDEIRKNAQLNIEL |
| | numbering corresponds | EAAHH |
| | to full length NAMPT, | |
| | e.g SEQ ID NO: 19) | |
| | (including HIS tag) | |
| SEQ ID NO: 10 | Truncated hNAMPTcif- | PAGNFVTLEEGKGDLEEYGQDLLHTVFKNGKVTKSY |
| | T4 (NAMPT₄₃₆₋₄₉₁, | SFDEIRKNAQLNIELEAAHH |
| | numbering corresponds | |
| | to full length NAMPT, | |
| | e.g SEQ ID NO: 19) | |
| SEQ ID NO: 11 | Truncated hNAMPTcif- | HHHHHHHHENLYFQGPAGNFVTLEEGKGDLEEYGQ |
| | T4 (NAMPT₄₃₆₋₄₉₁, | DLLHTVFKNGKVTKSYSFDEIRKNAQLNIELEAAHH |
| | numbering corresponds | |
| | to full length NAMPT, | |
| | e.g SEQ ID NO: 19) | |
| | (including HIS tag) | |
| SEQ ID NO: 12 | Human PIGF123-141 | RRRPKGRGKRRREKQRPTD |
| | (ECM-binding domain | |
| | derived from placenta | |
| | growth factor) | |
| SEQ ID NO: 13 | Human Areg25-42 | RKKKGGKNGKNRR |
| | (ECM-binding domain | |
| | derived from | |
| | amphiregulin) | |
| SEQ ID NO: 14 | Human von Willebrand | WREPSFMALS |
| | factor (vWF) ECM | |
| | binding moiety 1 | |
| SEQ ID NO: 15 | Human von Willebrand | CSQPLDVILLLDGSSSFPASYFDEMKSFAKAFISKANI |
| | factor (vWF) ECM | GPRLTQVSVLQYGSITTIDVPWNVVPEKAHLLSLVDV |
| | binding moiety 2 | MQREGGPSQIGDALGFAVRYLTSEMHGARPGASKA |
| | | VVILVTDVSVDSVDAAADAARSNRVTVFPIGIGDRYD |
| | | AAQLRILAGPAGDSNVVKLQRIEDLPTMVTLGNSFLH |
| | | KLCSGFVRICTG |
| SEQ ID NO: 16 | Human Collagenase | TKKTLRT |
| | ECM binding moiety | |
| SEQ ID NO: 17 | Human PIGF123- | RRRPKGRGKRRREKQRPTDCSYVVTNGLGVNVFKD |
| | 141/mouse NAMPTcif | PVADPNKRSKKGRLSLHRTPAGNFVTLEEGKGDLEE |
| | with Cys | YGHDLLHTVFKNGKVTKSYSFDEVRKNAQLNIEQDV |
| | | APH |
| SEQ ID NO: 18 | Human Areg25- | RKKKGGKNGKNRRCSYVVTNGLGVNVFKDPVADPN |
| | 42/mouse NAMPTcif | KRSKKGRLSLHRTPAGNFVTLEEGKGDLEEYGHDLL |
| | with Cys | HTVFKNGKVTKSYSFDEVRKNAQLNIEQDVAPH |
| SEQ ID NO: 19 | Example of Human | MNPAAEAEFNILLATDSYKVTHYKQYPPNTSKVYSYF |
| | NAMPT as CCR5 | ECREKKTENSKLRKVKYEETVFYGLQYILNKYLKGKV |
| | interacting polypeptide | VTKEKIQEAKDVYKEHFQDDVFNEKGWNYILEKYDG |
| | | HLPIEIKAVPEGFVIPRGNVLFTVENTDPECYWLTNWI |
| | | ETILVQSWYPITVATNSREQKKILAKYLLETSGNLDGL |
| | | EYKLHDFGYRGVSSQETAGIGASAHLVNFKGTDTVA |
| | | GLALIKKYYGTKDPVPGYSVPAAEHSTITAWGKDHE |
| | | KDAFEHIVTQFSSVPVSVVSDSYDIYNACEKIWGEDL |
| | | RHLIVSRSTQAPLIIRPDSGNPLDTVLKVLEILGKKFPV |
| | | TENSKGYKLLPPYLRVIQGDGVDINTLQEIVEGMKQK |
| | | MWSIENIAFGSGGGLLQKLTRDLLNCSFKCSYVVTN |
| | | GLGINVFKDPVADPNKRSKKGRLSLHRTPAGNFVTL |
| | | EEGKGDLEEYGQDLLHTVFKNGKVTKSYSFDEIRKN |
| | | AQLNIELEAAHH |
| SEQ ID NO: 20 | Example of Mouse | MNAAAEAEFNILLATDSYKVTHYKQYPPNTSKVYSYF |
| | NAMPT as CCR5 | ECREKKTENSKVRKVKYEETVFYGLQYILNKYLKGKV |
| | interacting polypeptide | VTKEKIQEAKEVYREHFQDDVFNERGWNYILEKYDG |
| | | HLPIEVKAVPEGSVIPRGNVLFTVENTDPECYWLTN |
| | | WIETILVQSWYPITVATNSREQKKILAKYLLETSGNLD |
| | | GLEYKLHDFGYRGVSSQETAGIGASAHLVNFKGTDT |
| | | VAGIALIKKYYGTKDPVPGYSVPAAEHSTITAWGKDH |
| | | EKDAFEHIVTQFSSVPVSVVSDSYDIYNACEKIWGED |
| | | LRHLIVSRSTEAPLIIRPDSGNPLDTVLKVLDILGKKFP |
| | | VTENSKGYKLLPPYLRVIQGDGVDINTLQEIVEGMKQ |
| | | KKWSIENVSFGSGGALLQKLTRDLLNCSFKCSYVVT |
| | | NGLGVNVFKDPVADPNKRSKKGRLSLHRTPAGNFV |
| | | TLEEGKGDLEEYGHDLLHTVFKNGKVTKSYSFDEVR |
| | | KNAQLNIEQDVAPH |
| SEQ ID NO: 21 | Example of a Human | agctatgttgtaactaatggccttgggattaacgtcttcaaggacccagttgc |
| | cDNA NAMPT cif | tgatcccaacaaaaggtccaaaaagggccgattatotttacataggacgc |
| | fragment encoding | cagcagggaattttgttacactggaggaaggaaaaggagaccttgagga |
| | sequence | atatggtcaggatcttctccatactgtottcaagaatggcaaggtgacaaa |
| | | aagctattcatttgatgaaataagaaaaaatgcacagctgaatattgaact |
| | | ggaagcagcacatcatta |
| SEQ ID NO: 22 | Mouse cDNA encoding | agctatgttgtaaccaatggccttggggttaatgtgtttaaggacccagttgc |
| | NAMPTcif fragment | tgatcccaacaaaaggtcaaaaaagggccggttatctttacataggacac |
| | | cagcggggaactttgttacacttgaagaaggaaaaggagaccttgagga |
| | | atatggccatgatcttctccatacggttttcaagaatgggaaggtgacaaa |
| | | aagctactcatttgatgaagtcagaaaaaatgcacagctgaacatcgag |
| | | caggacgtggcacctcatt |
| SEQ ID NO: 23 | Example of Human DNA | atgaatcctgcggcagaagccgagttcaacatcctcctggccaccgactc |
| | encoding NAMPT full | ctacaaggttactcactataaacaatatccacccaacacaagcaaagttt |
| | length | attcctactttgaatgccgtgaaaagaagacagaaaactccaaattaagg |
| | | aaggtgaaatatgaggaaacagtattttatgggttgcagtacattcttaata |
| | | agtacttaaaaggtaaagtagtaaccaaagagaaaatccaggaagcca |
| | | aagatgtctacaaagaacatttccaagatgatgtctttaatgaaaagggat |
| | | ggaactacattcttgagaagtatgatgggcatcttccaatagaaataaaag |
| | | ctgttcctgagggctttgtcattcccagaggaaatgttctcttcacggtggaa |
| | | aacacagatccagagtgttactggcttacaaattggattgagactattcttgt |
| | | tcagtcctggtatccaatcacagtggccacaaattctagagagcagaaga |
| | | aaatattggccaaatatttgttagaaacttctggtaacttagatggtctggaat |
| | | acaagttacatgattttggctacagaggagtctcttcccaagagactgctgg |
| | | cataggagcatctgctcacttggttaacttcaaaggaacagatacagtagc |
| | | aggacttgctctaattaaaaaatattatggaacgaaagatcctgttccagg |
| | | ctattctgttccagcagcagaacacagtaccataacagcttgggggaaag |
| | | accatgaaaaagatgcttttgaacatattgtaacacagttttcatcagtgcct |
| | | gtatctgtggtcagcgatagctatgacatttataatgcgtgtgagaaaatatg |
| | | gggtgaagatctaagacatttaatagtatcgagaagtacacaggcacca |
| | | ctaataatcagacctgattctggaaaccctcttgacactgtgttaaaggttttg |
| | | gagattttaggtaagaagtttcctgttactgagaactcaaagggttacaagtt |
| | | gctgccaccttatcttagagttattcaaggggatggagtagatattaatacct |
| | | tacaagagattgtagaaggcatgaaacaaaaaatgtggagtattgaaaa |
| | | tattgccttcggttctggtggaggtttgctacagaagttgacaagagatctctt |
| | | gaattgttccttcaagtgtagctatgttgtaactaatggccttgggattaacgt |
| | | cttcaaggacccagttgctgatcccaacaaaaggtccaaaaagggccg |
| | | attatotttacataggacgccagcagggaattttgttacactggaggaagg |
| | | aaaaggagaccttgaggaatatggtcaggatcttctccatactgtottcaa |
| | | gaatggcaaggtgacaaaaagctattcatttgatgaaataagaaaaaat |
| | | gcacagctgaatattgaactggaagcagcacatcatta |
| SEQ ID NO: 24 | Mouse cDNA encoding | atgaatgctgcggcagaagccgagttcaacatcctgctggccaccgactc |
| | NAMPT full-length | gtacaaggttactcactataaacaatacccacccaacacaagcaaagttt |
| | | attcctactttgaatgccgtgaaaagaagacagaaaactccaaagtaag |
| | | gaaggtgaaatacgaggaaacagtattttatgggttgcagtacattcttaat |
| | | aagtacttaaaaggtaaagtagtgaccaaagagaaaatccaggaggcc |
| | | aaagaagtgtacagagaacatttccaagatgatgtctttaacgaaagagg |
| | | atggaactacatccttgagaaatacgatggtcatctcccgattgaagtaaa |
| | | ggctgttcccgagggctctgtcatccccagagggaacgtgctgttcacagt |
| | | ggaaaacacagacccagagtgctactggcttaccaattggattgagacta |
| | | ttcttgttcagtcctggtatccaattacagtggccacaaattccagagaaca |
| | | gaagagaatactggccaaatatttgttagaaacctctggtaacttagatggt |
| | | ctggaatacaagttacatgactctggttacagaggagtctcttcgcaagag |
| | | actgctggcataggggcatctgctcatttggttaacttaaaaggaacagat |
| | | actgtggcgggaattgctctaattaaaaaatactatgggacaaaagatcct |
| | | gttccaggctattctgttccagcagcagagcacagtaccataacggcttgg |
| | | gggaaagaccatgagaaagatgcttttgaacacatagtaacacagttctc |
| | | atcagtgcctgtgtctgtggtcagcgatagctatgacatttataatgcgtgtg |
| | | agaaaatatggggtgaagacctgagacatctgatagtatcgagaagtac |
| | | agaggcaccactaatcatcagacctgactctggaaatcctcttgacactgt |
| | | attgaaggtcttagatattttaggcaagaagtttcctgttactgagaactcaa |
| | | aaggctacaagttgctgccaccttatcttagagtcattcaaggagatggcgt |
| | | ggatatcaatactttacaagagattgtagagggaatgaaacaaaagaag |
| | | tggagtatcgagaatgtctccttcggttctggtggcgctttgctacagaagtt |
| | | aacccgagacctcttgaattgctccttcaagtgcagctatgttgtaaccaat |
| | | ggccttggggttaatgtgtttaaggacccagttgctgatcccaacaaaagg |
| | | tcaaaaaagggccggttatctttacataggacaccagcggggaactttgtt |
| | | acacttgaagaaggaaaaggagaccttgaggaatatggccatgatcttct |
| | | ccatacggttttcaagaatgggaaggtgacaaaaagctactcatttgatga |
| | | agtcagaaaaaatgcacagctgaacatcgagcaggacgtggcacctca |
| | | tt |
| SEQ ID NO: 25 | Example of nucleotide | TTCAAAGACCCGGTTGCTGACCCGAACAAACGTTC |
| | sequence of | TAAAAAAGGTCGTCTGTCTCTGCACCGTACCCCGG |
| | hNAMPTcif-T1 | CTGGTAACTTCGTTACCCTGGAAGAAGGTAAAGGT |
| | | GACCTGGAAGAATACGGTCAGGACCTGCTGCACA |
| | | CCGTTTTCAAAAACGGTAAAGTTACCAAATCTTACT |
| | | CTTTCGACGAAATCCGTAAAAACGCTCAGCTGAAC |
| | | ATCGAACTGGAAGCTGCTCACCACTAA |
| SEQ ID NO: 26 | Example of nucleotide | AACAAACGTTCTAAAAAAGGTCGTCTGTCTCTGCA |
| | sequence of | CCGTACCCCGGCTGGTAACTTCGTTACCCTGGAA |
| | hNAMPTcif-T2 | GAAGGTAAAGGTGACCTGGAAGAATACGGTCAGG |
| | | ACCTGCTGCACACCGTTTTCAAAAACGGTAAAGTT |
| | | ACCAAATCTTACTCTTTCGACGAAATCCGTAAAAAC |
| | | GCTCAGCTGAACATCGAACTGGAAGCTGCTCACC |
| | | ACTAA |
| SEQ ID NO: 27 | Example of nucleotide | CTGTCTCTGCACCGTACCCCGGCTGGTAACTTCGT |
| | sequence of | TACCCTGGAAGAAGGTAAAGGTGACCTGGAAGAA |
| | hNAMPTcif-T3 | TACGGTCAGGACCTGCTGCACACCGTTTTCAAAAA |
| | | CGGTAAAGTTACCAAATCTTACTCTTTCGACGAAAT |
| | | CCGTAAAAACGCTCAGCTGAACATCGAACTGGAA |
| | | GCTGCTCACCACTAA |
| SEQ ID NO: 28 | Example of nucleotide | CCGGCTGGTAACTTCGTTACCCTGGAAGAAGGTA |
| | sequence of | AAGGTGACCTGGAAGAATACGGTCAGGACCTGCT |
| | hNAMPTcif-T4 | GCACACCGTTTTCAAAAACGGTAAAGTTACCAAAT |
| | | CTTACTCTTTCGACGAAATCCGTAAAAACGCTCAG |
| | | CTGAACATCGAACTGGAAGCTGCTCACCACTAA |
| SEQ ID NO: 29 | Example of nucleotide | TGGCGCGAACCGAGCTTTATGGCGCTGAGCTCTT |
| | sequence of hvWF1- | ACGTTGTTACCAACGGTCTGGGTATCAACGTTTTC |
| | hNAMPTcif | AAAGACCCGGTTGCTGACCCGAACAAACGTTCTAA |
| | | AAAAGGTCGTCTGTCTCTGCACCGTACCCCGGCT |
| | | GGTAACTTCGTTACCCTGGAAGAAGGTAAAGGTGA |
| | | CCTGGAAGAATACGGTCAGGACCTGCTGCACACC |
| | | GTTTTCAAAAACGGTAAAGTTACCAAATCTTACTCT |
| | | TTCGACGAAATCCGTAAAAACGCTCAGCTGAACAT |
| | | CGAACTGGAAGCTGCTCACCACTAA |
| SEQ ID NO: 30 | Example of nucleotide | TGCAGCCAGCCGCTGGATGTGATTCTGCTGCTGG |
| | sequence of hvWF2- | ATGGCAGCAGCAGCTTTCCGGCGAGCTATTTTGAT |
| | hNAMPTcif | GAAATGAAAAGCTTTGCGAAAGCGTTTATTAGCAA |
| | | AGCGAACATTGGCCCGCGCCTGACCCAGGTGAGC |
| | | GTGCTGCAGTATGGCAGCATTACCACCATTGATGT |
| | | GCCGTGGAACGTGGTGCCGGAAAAAGCGCATCTG |
| | | CTGAGCCTGGTGGATGTGATGCAGCGCGAAGGCG |
| | | GCCCGAGCCAGATTGGCGATGCGCTGGGCTTTGC |
| | | GGTGCGCTATCTGACCAGCGAAATGCATGGCGCG |
| | | CGCCCGGGCGCGAGCAAAGCGGTGGTGATTCTG |
| | | GTGACCGATGTGAGCGTGGATAGCGTGGATGCGG |
| | | CGGCGGATGCGGCGCGCAGCAACCGCGTGACCG |
| | | TGTTTCCGATTGGCATTGGCGATCGCTATGATGCG |
| | | GCGCAGCTGCGCATTCTGGCGGGCCCGGCGGGC |
| | | GATAGCAACGTGGTGAAACTGCAGCGCATTGAAG |
| | | ATCTGCCGACCATGGTGACCCTGGGCAACAGCTT |
| | | TCTGCATAAACTGTGCAGCGGCTTTGTGCGCATTT |
| | | GCACCGGCTCTTACGTTGTTACCAACGGTCTGGG |
| | | TATCAACGTTTTCAAAGACCCGGTTGCTGACCCGA |
| | | ACAAACGTTCTAAAAAAGGTCGTCTGTCTCTGCAC |
| | | CGTACCCCGGCTGGTAACTTCGTTACCCTGGAAG |
| | | AAGGTAAAGGTGACCTGGAAGAATACGGTCAGGA |
| | | CCTGCTGCACACCGTTTTCAAAAACGGTAAAGTTA |
| | | CCAAATCTTACTCTTTCGACGAAATCCGTAAAAAC |
| | | GCTCAGCTGAACATCGAACTGGAAGCTGCTCACC |
| | | ACTAA |
| SEQ ID NO: 31 | hCol-hNAMPTcif | ACCAAAAAAACCCTGCGCACCTCTTACGTTGTTAC |
| | | CAACGGTCTGGGTATCAACGTTTTCAAAGACCCGG |
| | | TTGCTGACCCGAACAAACGTTCTAAAAAAGGTCGT |
| | | CTGTCTCTGCACCGTACCCCGGCTGGTAACTTCGT |
| | | TACCCTGGAAGAAGGTAAAGGTGACCTGGAAGAA |
| | | TACGGTCAGGACCTGCTGCACACCGTTTTCAAAAA |
| | | CGGTAAAGTTACCAAATCTTACTCTTTCGACGAAAT |
| | | CCGTAAAAACGCTCAGCTGAACATCGAACTGGAA |
| | | GCTGCTCACCACTAA |
| SEQ ID NO: 32 | Example nucleotide | TCTTACGTTGTTACCAACGGTCTGGGTATCAACGT |
| | sequence of hNAMPTcif | TTTCAAAGACCCGGTTGCTGACCCGAACAAACGTT |
| | | CTAAAAAAGGTCGTCTGTCTCTGCACCGTACCCCG |
| | | GCTGGTAACTTCGTTACCCTGGAAGAAGGTAAAG |
| | | GTGACCTGGAAGAATACGGTCAGGACCTGCTGCA |
| | | CACCGTTTTCAAAAACGGTAAAGTTACCAAATCTTA |
| | | CTCTTTCGACGAAATCCGTAAAAACGCTCAGCTGA |
| | | ACATCGAACTGGAAGCTGCTCACCACTAA |
| SEQ ID NO: 33 | Truncated hNAMPTcif- | NKRSKKGRLSLHRTPAGNFVTLEEGKGDLEEYGQDL |
| | T5 (NAMPT₄₂₂₋₄₇₁, | LHTVFKNGKVTKS |
| | numbering corresponds | |
| | to full length NAMPT, | |
| | e.g SEQ ID NO: 19) | |
| SEQ ID NO: 34 | Truncated hNAMPTcif- | HHHHHHHHENLYFQGNKRSKKGRLSLHRTPAGNFV |
| | T5 (NAMPT₄₂₂₋₄₇₁, | TLEEGKGDLEEYGQDLLHTVFKNGKVTKS |
| | numbering corresponds | |
| | to full length NAMPT, | |
| | e.g SEQ ID NO: 19) | |
| | (including HIS tag) | |
| SEQ ID NO: 35 | Example of nucleotide | AACAAACGTTCTAAAAAAGGTCGTCTGTCTCTGCA |
| | sequence of | CCGTACCCCGGCTGGTAACTTCGTTACCCTGGAA |
| | hNAMPTcif-T5 | GAAGGTAAAGGTGACCTGGAAGAATACGGTCAGG |
| | | ACCTGCTGCACACCGTTTTCAAAAACGGTAAAGTT |
| | | ACCAAATCTTAC |
| SEQ ID NO: 36 | Example of IL-2 signal | MYRMQLLSCIALSLALVTNS |
| | sequence | |
| SEQ ID NO: 37 | Example of IgG1 Fc with | MYRMQLLSCIALSLALVTNSCPPCPAPELLGGPSVFL |
| | IL-2 sequence | FPPKPKDTLMISRTPEVTCVVVDVSHEDPEVKFNWY |
| | | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGK |
| SEQ ID NO: 38 | Example of IgG1 Fc | CPPCPAPELLGGPSVFLFPPKPKDTLMISRTPEVTCV |
| | | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGK |
| SEQ ID NO: 39 | Example of IgG1 Fc with | MYRMQLLSCIALSLALVTNSCPPCPAPEAAGGPSVF |
| | IL-2 sequence and | LFPPKPKDTLYITREPEVTCVVVDVSHEDPEVKFNW |
| | LALA/YTE mutations | YVDGVEVHNAKTKPREEQYNSTYRVVSVLTVLHQD |
| | (underlined and bolded) | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| SEQ ID NO: 40 | Example of IgG1 Fc with | CPPCPAPEAAGGPSVFLFPPKPKDTLYITREPEVTCV |
| | LALA/YTE mutations | VVDVSHEDPEVKFNWYVDGVEVHNAKTKPREEQYN |
| | (underlined and bolded) | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGK |
| SEQ ID NO: 41 | Example of linker | GGGGSGGGGSGGGGS |
| SEQ ID NO: 42 | Example fusion protein | CPPCPAPELLGGPSVFLFPPKPKDTLMISRTPEVTCV |
| | IgG1 Fc-NAMPT 422-491 | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSNKRSKKGRLSLHR |
| | | TPAGNFVTLEEGKGDLEEYGQDLLHTVFKNGKVTKS |
| | | YSFDEIRKNAQLNIELEAAHH |
| SEQ ID NO: 43 | Example fusion protein | MYRMQLLSCIALSLALVTNSCPPCPAPELLGGPSVFL |
| | IgG1 Fc-NAMPT422-491 | FPPKPKDTLMISRTPEVTCVVVDVSHEDPEVKFNWY |
| | with IL-2 signal peptide | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGG |
| | | GSGGGGSNKRSKKGRLSLHRTPAGNFVTLEEGKGD |
| | | LEEYGQDLLHTVFKNGKVTKSYSFDEIRKNAQLNIEL |
| | | EAAHH |
| SEQ ID NO: 44 | Example fusion protein | CPPCPAPEAAGGPSVFLFPPKPKDTLYITREPEVTCV |
| | IgG1 Fc-NAMPT422-491 | VVDVSHEDPEVKFNWYVDGVEVHNAKTKPREEQYN |
| | with LALA/YTE | STYRVVSVLTVLHQDWLNGKEYKCKVSNKALPAPIE |
| | mutations | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSNKRSKKGRLSLHR |
| | | TPAGNFVTLEEGKGDLEEYGQDLLHTVFKNGKVTKS |
| | | YSFDEIRKNAQLNIELEAAHH |
| SEQ ID NO: 45 | Example fusion protein | MYRMQLLSCIALSLALVTNSCPPCPAPEAAGGPSVF |
| | IgG1 Fc-NAMPT422-491 | LFPPKPKDTLYITREPEVTCVVVDVSHEDPEVKFNW |
| | with IL-2 signal peptide | YVDGVEVHNAKTKPREEQYNSTYRVVSVLTVLHQD |
| | and LALA/YTE | WLNGKEYKCKVSNKALPAPIEKTISKAKGQPREPQV |
| | mutations | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGKGGGGSGG |
| | | GGSGGGGSNKRSKKGRLSLHRTPAGNFVTLEEGKG |
| | | DLEEYGQDLLHTVFKNGKVTKSYSFDEIRKNAQLNIE |
| | | LEAAHH |
| SEQ ID NO: 46 | Example fusion protein | CPPCPAPELLGGPSVFLFPPKPKDTLMISRTPEVTCV |
| | IgG1 Fc-NAMPT₄₀₂₋₄₉₁ | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSSYVVTNGLGINVFK |
| | | DPVADPNKRSKKGRLSLHRTPAGNFVTLEEGKGDLE |
| | | EYGQDLLHTVFKNGKVTKSYSFDEIRKNAQLNIELEA |
| | | AHH |
| SEQ ID NO: 47 | Example fusion protein | MYRMQLLSCIALSLALVTNSCPPCPAPELLGGPSVFL |
| | IgG1 Fc-NAMPT₄₀₂₋₄₉₁ | FPPKPKDTLMISRTPEVTCVVVDVSHEDPEVKFNWY |
| | with IL-2 signal peptide | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGG |
| | | GSGGGGSSYVVTNGLGINVFKDPVADPNKRSKKGR |
| | | LSLHRTPAGNFVTLEEGKGDLEEYGQDLLHTVFKNG |
| | | KVTKSYSFDEIRKNAQLNIELEAAHH |
| SEQ ID NO: 48 | Example fusion protein | CPPCPAPEAAGGPSVFLFPPKPKDTLYITREPEVTCV |
| | IgG1 Fc-NAMPT₄₀₂₋₄₉₁ | VVDVSHEDPEVKFNWYVDGVEVHNAKTKPREEQYN |
| | with LALA/YTE | STYRVVSVLTVLHQDWLNGKEYKCKVSNKALPAPIE |
| | mutations | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSSYVVTNGLGINVFK |
| | | DPVADPNKRSKKGRLSLHRTPAGNFVTLEEGKGDLE |
| | | EYGQDLLHTVFKNGKVTKSYSFDEIRKNAQLNIELEA |
| | | AHH |
| SEQ ID NO: 49 | Example fusion protein | MYRMQLLSCIALSLALVTNSCPPCPAPEAAGGPSVF |
| | IgG1 Fc-NAMPT₄₀₂₋₄₉₁ | LFPPKPKDTLYITREPEVTCVVVDVSHEDPEVKFNW |
| | with IL-2 signal peptide | YVDGVEVHNAKTKPREEQYNSTYRVVSVLTVLHQD |
| | and LALA/YTE | WLNGKEYKCKVSNKALPAPIEKTISKAKGQPREPQV |
| | mutations | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGKGGGGSGG |
| | | GGSGGGGSSYVVTNGLGINVFKDPVADPNKRSKKG |
| | | RLSLHRTPAGNFVTLEEGKGDLEEYGQDLLHTVFKN |
| | | GKVTKSYSFDEIRKNAQLNIELEAAHH |
| SEQ ID NO: 50 | Example fusion protein | CPPCPAPELLGGPSVFLFPPKPKDTLMISRTPEVTCV |
| | IgG1 Fc-NAMPT414-491 | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSFKDPVADPNKRSK |
| | | KGRLSLHRTPAGNFVTLEEGKGDLEEYGQDLLHTVF |
| | | KNGKVTKSYSFDEIRKNAQLNIELEAAHH |
| SEQ ID NO: 51 | Example fusion protein | MYRMQLLSCIALSLALVTNSCPPCPAPELLGGPSVFL |
| | IgG1 Fc-NAMPT414-491 | FPPKPKDTLMISRTPEVTCVVVDVSHEDPEVKFNWY |
| | with IL-2 signal peptide | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGG |
| | | GSGGGGSFKDPVADPNKRSKKGRLSLHRTPAGNFV |
| | | TLEEGKGDLEEYGQDLLHTVFKNGKVTKSYSFDEIR |
| | | KNAQLNIELEAAHH |
| SEQ ID NO: 52 | Example fusion protein | CPPCPAPEAAGGPSVFLFPPKPKDTLYITREPEVTCV |
| | IgG1 Fc-NAMPT414-491 | VVDVSHEDPEVKFNWYVDGVEVHNAKTKPREEQYN |
| | with LALA/YTE | STYRVVSVLTVLHQDWLNGKEYKCKVSNKALPAPIE |
| | mutations | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSFKDPVADPNKRSK |
| | | KGRLSLHRTPAGNFVTLEEGKGDLEEYGQDLLHTVF |
| | | KNGKVTKSYSFDEIRKNAQLNIELEAAHH |
| SEQ ID NO: 53 | Example fusion protein | MYRMQLLSCIALSLALVTNSCPPCPAPEAAGGPSVF |
| | IgG1 Fc-NAMPT414-491 | LFPPKPKDTLYITREPEVTCVVVDVSHEDPEVKFNW |
| | with IL-2 signal peptide | YVDGVEVHNAKTKPREEQYNSTYRVVSVLTVLHQD |
| | and LALA/YTE | WLNGKEYKCKVSNKALPAPIEKTISKAKGQPREPQV |
| | mutations | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGKGGGGSGG |
| | | GGSGGGGSFKDPVADPNKRSKKGRLSLHRTPAGNF |
| | | VTLEEGKGDLEEYGQDLLHTVFKNGKVTKSYSFDEI |
| | | RKNAQLNIELEAAHH |
| SEQ ID NO: 54 | Example fusion protein | CPPCPAPELLGGPSVFLFPPKPKDTLMISRTPEVTCV |
| | IgG1 Fc-NAMPT 430-491 | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSLSLHRTPAGNFVT |
| | | LEEGKGDLEEYGQDLLHTVFKNGKVTKSYSFDEIRK |
| | | NAQLNIELEAAHH |
| SEQ ID NO: 55 | Example fusion protein | MYRMQLLSCIALSLALVTNSCPPCPAPELLGGPSVFL |
| | IgG1 Fc-NAMPT 430-491 | FPPKPKDTLMISRTPEVTCVVVDVSHEDPEVKFNWY |
| | with IL-2 signal peptide | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGG |
| | | GSGGGGSLSLHRTPAGNFVTLEEGKGDLEEYGQDL |
| | | LHTVFKNGKVTKSYSFDEIRKNAQLNIELEAAHH |
| SEQ ID NO: 56 | Example fusion protein | CPPCPAPEAAGGPSVFLFPPKPKDTLYITREPEVTCV |
| | IgG1 Fc-NAMPT430-491 | VVDVSHEDPEVKFNWYVDGVEVHNAKTKPREEQYN |
| | with LALA/YTE | STYRVVSVLTVLHQDWLNGKEYKCKVSNKALPAPIE |
| | mutations | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSLSLHRTPAGNFVT |
| | | LEEGKGDLEEYGQDLLHTVFKNGKVTKSYSFDEIRK |
| | | NAQLNIELEAAHH |
| SEQ ID NO: 57 | Example fusion protein | MYRMQLLSCIALSLALVTNSCPPCPAPEAAGGPSVF |
| | IgG1 Fc-NAMPT 430-491 | LFPPKPKDTLYITREPEVTCVVVDVSHEDPEVKFNW |
| | with IL-2 signal peptide | YVDGVEVHNAKTKPREEQYNSTYRVVSVLTVLHQD |
| | and LALA/YTE | WLNGKEYKCKVSNKALPAPIEKTISKAKGQPREPQV |
| | mutations | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGKGGGGSGG |
| | | GGSGGGGSLSLHRTPAGNFVTLEEGKGDLEEYGQD |
| | | LLHTVFKNGKVTKSYSFDEIRKNAQLNIELEAAHH |
| SEQ ID NO: 58 | Example fusion protein | CPPCPAPELLGGPSVFLFPPKPKDTLMISRTPEVTCV |
| | IgG1 Fc-NAMPT436-491 | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSPAGNFVTLEEGKG |
| | | DLEEYGQDLLHTVFKNGKVTKSYSFDEIRKNAQLNIE |
| | | LEAAHH |
| SEQ ID NO: 59 | Example fusion protein | MYRMQLLSCIALSLALVTNSCPPCPAPELLGGPSVFL |
| | IgG1 Fc-NAMPT 436-491 | FPPKPKDTLMISRTPEVTCVVVDVSHEDPEVKFNWY |
| | with IL-2 signal peptide | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGG |
| | | GSGGGGSPAGNFVTLEEGKGDLEEYGQDLLHTVFK |
| | | NGKVTKSYSFDEIRKNAQLNIELEAAHH |
| SEQ ID NO: 60 | Example fusion protein | CPPCPAPEAAGGPSVFLFPPKPKDTLYITREPEVTCV |
| | IgG1 Fc-NAMPT436-491 | VVDVSHEDPEVKFNWYVDGVEVHNAKTKPREEQYN |
| | with LALA/YTE | STYRVVSVLTVLHQDWLNGKEYKCKVSNKALPAPIE |
| | mutations | KTISKAKGQPREPQVYTLPPSRDELTKNQVSLTCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKGGGGSGGGGSGGGGSPAGNFVTLEEGKG |
| | | DLEEYGQDLLHTVFKNGKVTKSYSFDEIRKNAQLNIE |
| | | LEAAHH |
| SEQ ID NO: 61 | Example fusion protein | MYRMQLLSCIALSLALVTNSCPPCPAPEAAGGPSVF |
| | IgG1 Fc-NAMPT436-491 | LFPPKPKDTLYITREPEVTCVVVDVSHEDPEVKFNW |
| | with IL-2 signal peptide | YVDGVEVHNAKTKPREEQYNSTYRVVSVLTVLHQD |
| | and LALA/YTE | WLNGKEYKCKVSNKALPAPIEKTISKAKGQPREPQV |
| | mutations | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGKGGGGSGG |
| | | GGSGGGGSPAGNFVTLEEGKGDLEEYGQDLLHTVF |
| | | KNGKVTKSYSFDEIRKNAQLNIELEAAHH |
| SEQ ID NO: 62 | Example nucleotide | ATGTACAGGATGCAGCTGCTGAGCTGCATCGCCC |
| | sequence for IL-2 signal | TGAGCCTGGCCCTGGTGACCAACAGC |
| | sequence | |
| SEQ ID NO: 63 | Example nucleotide | ATGTACAGGATGCAGCTGCTGAGCTGCATCGCCC |
| | sequence for IgG1 Fc | TGAGCCTGGCCCTGGTGACCAACAGCTGCCCCCC |
| | with IL-2 sequence | CTGCCCCGCCCCCGAGCTGCTGGGCGGCCCCAG |
| | | CGTGTTCCTGTTCCCCCCCAAGCCCAAGGACACC |
| | | CTGATGATCAGCAGGACCCCCGAGGTGACCTGCG |
| | | TGGTGGTGGACGTGAGCCACGAGGACCCCGAGG |
| | | TGAAGTTCAACTGGTACGTGGACGGCGTGGAGGT |
| | | GCACAACGCCAAGACCAAGCCCAGGGAGGAGCA |
| | | GTACAACAGCACCTACAGGGTGGTGAGCGTGCTG |
| | | ACCGTGCTGCACCAGGACTGGCTGAACGGCAAGG |
| | | AGTACAAGTGCAAGGTGAGCAACAAGGCCCTGCC |
| | | CGCCCCCATCGAGAAGACCATCAGCAAGGCCAAG |
| | | GGCCAGCCCAGGGAGCCCCAGGTGTACACCCTG |
| | | CCCCCCAGCAGGGACGAGCTGACCAAGAACCAG |
| | | GTGAGCCTGACCTGCCTGGTGAAGGGCTTCTACC |
| | | CCAGCGACATCGCCGTGGAGTGGGAGAGCAACG |
| | | GCCAGCCCGAGAACAACTACAAGACCACCCCCCC |
| | | CGTGCTGGACAGCGACGGCAGCTTCTTCCTGTAC |
| | | AGCAAGCTGACCGTGGACAAGAGCAGGTGGCAGC |
| | | AGGGCAACGTGTTCAGCTGCAGCGTGATGCACGA |
| | | GGCCCTGCACAACCACTACACCCAGAAGAGCCTG |
| | | AGCCTGAGCCCCGGCAAG |
| SEQ ID NO: 64 | Example nucleotide | TGCCCCCCCTGCCCCGCCCCCGAGCTGCTGGGC |
| | sequence for IgG1 Fc | GGCCCCAGCGTGTTCCTGTTCCCCCCCAAGCCCA |
| | | AGGACACCCTGATGATCAGCAGGACCCCCGAGGT |
| | | GACCTGCGTGGTGGTGGACGTGAGCCACGAGGA |
| | | CCCCGAGGTGAAGTTCAACTGGTACGTGGACGGC |
| | | GTGGAGGTGCACAACGCCAAGACCAAGCCCAGG |
| | | GAGGAGCAGTACAACAGCACCTACAGGGTGGTGA |
| | | GCGTGCTGACCGTGCTGCACCAGGACTGGCTGAA |
| | | CGGCAAGGAGTACAAGTGCAAGGTGAGCAACAAG |
| | | GCCCTGCCCGCCCCCATCGAGAAGACCATCAGCA |
| | | AGGCCAAGGGCCAGCCCAGGGAGCCCCAGGTGT |
| | | ACACCCTGCCCCCCAGCAGGGACGAGCTGACCAA |
| | | GAACCAGGTGAGCCTGACCTGCCTGGTGAAGGGC |
| | | TTCTACCCCAGCGACATCGCCGTGGAGTGGGAGA |
| | | GCAACGGCCAGCCCGAGAACAACTACAAGACCAC |
| | | CCCCCCCGTGCTGGACAGCGACGGCAGCTTCTTC |
| | | CTGTACAGCAAGCTGACCGTGGACAAGAGCAGGT |
| | | GGCAGCAGGGCAACGTGTTCAGCTGCAGCGTGAT |
| | | GCACGAGGCCCTGCACAACCACTACACCCAGAAG |
| | | AGCCTGAGCCTGAGCCCCGGCAAG |
| SEQ ID NO: 65 | Example nucleotide | ATGTACAGGATGCAGCTGCTGAGCTGCATCGCCC |
| | sequence for IgG1 Fc | TGAGCCTGGCCCTGGTGACCAACAGCTGCCCCCC |
| | with IL-2 sequence and | CTGCCCCGCCCCCGAGGCCGCCGGCGGCCCCAG |
| | LALA/YTE mutations | CGTGTTCCTGTTCCCCCCCAAGCCCAAGGACACC |
| | (underlined and bolded) | CTGTACATCACCAGGGAGCCCGAGGTGACCTGCG |
| | | TGGTGGTGGACGTGAGCCACGAGGACCCCGAGG |
| | | TGAAGTTCAACTGGTACGTGGACGGCGTGGAGGT |
| | | GCACAACGCCAAGACCAAGCCCAGGGAGGAGCA |
| | | GTACAACAGCACCTACAGGGTGGTGAGCGTGCTG |
| | | ACCGTGCTGCACCAGGACTGGCTGAACGGCAAGG |
| | | AGTACAAGTGCAAGGTGAGCAACAAGGCCCTGCC |
| | | CGCCCCCATCGAGAAGACCATCAGCAAGGCCAAG |
| | | GGCCAGCCCAGGGAGCCCCAGGTGTACACCCTG |
| | | CCCCCCAGCAGGGACGAGCTGACCAAGAACCAG |
| | | GTGAGCCTGACCTGCCTGGTGAAGGGCTTCTACC |
| | | CCAGCGACATCGCCGTGGAGTGGGAGAGCAACG |
| | | GCCAGCCCGAGAACAACTACAAGACCACCCCCCC |
| | | CGTGCTGGACAGCGACGGCAGCTTCTTCCTGTAC |
| | | AGCAAGCTGACCGTGGACAAGAGCAGGTGGCAGC |
| | | AGGGCAACGTGTTCAGCTGCAGCGTGATGCACGA |
| | | GGCCCTGCACAACCACTACACCCAGAAGAGCCTG |
| | | AGCCTGAGCCCCGGCAAG |
| SEQ ID NO: 66 | Example nucleotide | TGCCCCCCCTGCCCCGCCCCCGAGGCCGCCGGC |
| | sequence for IgG1 Fc | GGCCCCAGCGTGTTCCTGTTCCCCCCCAAGCCCA |
| | with LALA/YTE | AGGACACCCTGTACATCACCAGGGAGCCCGAGGT |
| | mutations (underlined | GACCTGCGTGGTGGTGGACGTGAGCCACGAGGA |
| | and bolded) | CCCCGAGGTGAAGTTCAACTGGTACGTGGACGGC |
| | | GTGGAGGTGCACAACGCCAAGACCAAGCCCAGG |
| | | GAGGAGCAGTACAACAGCACCTACAGGGTGGTGA |
| | | GCGTGCTGACCGTGCTGCACCAGGACTGGCTGAA |
| | | CGGCAAGGAGTACAAGTGCAAGGTGAGCAACAAG |
| | | GCCCTGCCCGCCCCCATCGAGAAGACCATCAGCA |
| | | AGGCCAAGGGCCAGCCCAGGGAGCCCCAGGTGT |
| | | ACACCCTGCCCCCCAGCAGGGACGAGCTGACCAA |
| | | GAACCAGGTGAGCCTGACCTGCCTGGTGAAGGGC |
| | | TTCTACCCCAGCGACATCGCCGTGGAGTGGGAGA |
| | | GCAACGGCCAGCCCGAGAACAACTACAAGACCAC |
| | | CCCCCCCGTGCTGGACAGCGACGGCAGCTTCTTC |
| | | CTGTACAGCAAGCTGACCGTGGACAAGAGCAGGT |
| | | GGCAGCAGGGCAACGTGTTCAGCTGCAGCGTGAT |
| | | GCACGAGGCCCTGCACAACCACTACACCCAGAAG |
| | | AGCCTGAGCCTGAGCCCCGGCAAG |
| SEQ ID NO: 67 | Example nucleotide | GGCGGCGGCGGCAGCGGCGGCGGCGGCAGCGG |
| | sequence for linker | CGGCGGCGGCAGC |
| SEQ ID NO: 68 | Example nucleotide | TGCCCCCCCTGCCCCGCCCCCGAGCTGCTGGGC |
| | sequence for fusion | GGCCCCAGCGTGTTCCTGTTCCCCCCCAAGCCCA |
| | protein IgG1 Fc- | AGGACACCCTGATGATCAGCAGGACCCCCGAGGT |
| | NAMPT422-491 | GACCTGCGTGGTGGTGGACGTGAGCCACGAGGA |
| | | CCCCGAGGTGAAGTTCAACTGGTACGTGGACGGC |
| | | GTGGAGGTGCACAACGCCAAGACCAAGCCCAGG |
| | | GAGGAGCAGTACAACAGCACCTACAGGGTGGTGA |
| | | GCGTGCTGACCGTGCTGCACCAGGACTGGCTGAA |
| | | CGGCAAGGAGTACAAGTGCAAGGTGAGCAACAAG |
| | | GCCCTGCCCGCCCCCATCGAGAAGACCATCAGCA |
| | | AGGCCAAGGGCCAGCCCAGGGAGCCCCAGGTGT |
| | | ACACCCTGCCCCCCAGCAGGGACGAGCTGACCAA |
| | | GAACCAGGTGAGCCTGACCTGCCTGGTGAAGGGC |
| | | TTCTACCCCAGCGACATCGCCGTGGAGTGGGAGA |
| | | GCAACGGCCAGCCCGAGAACAACTACAAGACCAC |
| | | CCCCCCCGTGCTGGACAGCGACGGCAGCTTCTTC |
| | | CTGTACAGCAAGCTGACCGTGGACAAGAGCAGGT |
| | | GGCAGCAGGGCAACGTGTTCAGCTGCAGCGTGAT |
| | | GCACGAGGCCCTGCACAACCACTACACCCAGAAG |
| | | AGCCTGAGCCTGAGCCCCGGCAAGGGCGGCGGC |
| | | GGCAGCGGCGGCGGCGGCAGCGGCGGCGGCGG |
| | | CAGCAACAAGAGGAGCAAGAAGGGCAGGCTGAG |
| | | CCTGCACAGGACCCCCGCCGGCAACTTCGTGACC |
| | | CTGGAGGAGGGCAAGGGCGACCTGGAGGAGTAC |
| | | GGCCAGGACCTGCTGCACACCGTGTTCAAGAACG |
| | | GCAAGGTGACCAAGAGCTACAGCTTCGACGAGAT |
| | | CAGGAAGAACGCCCAGCTGAACATCGAGCTGGAG |
| | | GCCGCCCACCACTGA |
| SEQ ID NO: 69 | Example nucleotide | TGCCCCCCCTGCCCCGCCCCCGAGCTGCTGGGC |
| | sequence for fusion | GGCCCCAGCGTGTTCCTGTTCCCCCCCAAGCCCA |
| | protein IgG1 Fc- | AGGACACCCTGATGATCAGCAGGACCCCCGAGGT |
| | NAMPT422-491 with IL-2 | GACCTGCGTGGTGGTGGACGTGAGCCACGAGGA |
| | signal peptide | CCCCGAGGTGAAGTTCAACTGGTACGTGGACGGC |
| | | GTGGAGGTGCACAACGCCAAGACCAAGCCCAGG |
| | | GAGGAGCAGTACAACAGCACCTACAGGGTGGTGA |
| | | GCGTGCTGACCGTGCTGCACCAGGACTGGCTGAA |
| | | CGGCAAGGAGTACAAGTGCAAGGTGAGCAACAAG |
| | | GCCCTGCCCGCCCCCATCGAGAAGACCATCAGCA |
| | | AGGCCAAGGGCCAGCCCAGGGAGCCCCAGGTGT |
| | | ACACCCTGCCCCCCAGCAGGGACGAGCTGACCAA |
| | | GAACCAGGTGAGCCTGACCTGCCTGGTGAAGGGC |
| | | TTCTACCCCAGCGACATCGCCGTGGAGTGGGAGA |
| | | GCAACGGCCAGCCCGAGAACAACTACAAGACCAC |
| | Example nucleotide | CCCCCCCGTGCTGGACAGCGACGGCAGCTTCTTC |
| | sequence for fusion | CTGTACAGCAAGCTGACCGTGGACAAGAGCAGGT |
| | protein IgG1 Fc- | GGCAGCAGGGCAACGTGTTCAGCTGCAGCGTGAT |
| | | GCACGAGGCCCTGCACAACCACTACACCCAGAAG |
| | | AGCCTGAGCCTGAGCCCCGGCAAGGGCGGCGGC |
| | | GGCAGCGGCGGCGGCGGCAGCGGCGGCGGCGG |
| | | CAGCAACAAGAGGAGCAAGAAGGGCAGGCTGAG |
| | | CCTGCACAGGACCCCCGCCGGCAACTTCGTGACC |
| | | CTGGAGGAGGGCAAGGGCGACCTGGAGGAGTAC |
| | | GGCCAGGACCTGCTGCACACCGTGTTCAAGAACG |
| | | GCAAGGTGACCAAGAGCTACAGCTTCGACGAGAT |
| | | CAGGAAGAACGCCCAGCTGAACATCGAGCTGGAG |
| | | GCCGCCCACCACTGA |
| SEQ ID NO: 70 | NAMPT 422-491 with | TGCCCCCCCTGCCCCGCCCCCGAGGCCGCCGGC |
| | LALA/YTE mutations | GGCCCCAGCGTGTTCCTGTTCCCCCCCAAGCCCA |
| | (underlined and bolded) | AGGACACCCTGTACATCACCAGGGAGCCCGAGGT |
| | | GACCTGCGTGGTGGTGGACGTGAGCCACGAGGA |
| | | CCCCGAGGTGAAGTTCAACTGGTACGTGGACGGC |
| | | GTGGAGGTGCACAACGCCAAGACCAAGCCCAGG |
| | | GAGGAGCAGTACAACAGCACCTACAGGGTGGTGA |
| | | GCGTGCTGACCGTGCTGCACCAGGACTGGCTGAA |
| | | CGGCAAGGAGTACAAGTGCAAGGTGAGCAACAAG |
| | | GCCCTGCCCGCCCCCATCGAGAAGACCATCAGCA |
| | | AGGCCAAGGGCCAGCCCAGGGAGCCCCAGGTGT |
| | | ACACCCTGCCCCCCAGCAGGGACGAGCTGACCAA |
| | | GAACCAGGTGAGCCTGACCTGCCTGGTGAAGGGC |
| | | TTCTACCCCAGCGACATCGCCGTGGAGTGGGAGA |
| | | GCAACGGCCAGCCCGAGAACAACTACAAGACCAC |
| | | CCCCCCCGTGCTGGACAGCGACGGCAGCTTCTTC |
| | | CTGTACAGCAAGCTGACCGTGGACAAGAGCAGGT |
| | | GGCAGCAGGGCAACGTGTTCAGCTGCAGCGTGAT |
| | | GCACGAGGCCCTGCACAACCACTACACCCAGAAG |
| | | AGCCTGAGCCTGAGCCCCGGCAAGGGCGGCGGC |
| | | GGCAGCGGCGGCGGCGGCAGCGGCGGCGGCGG |
| | | CAGCAACAAGAGGAGCAAGAAGGGCAGGCTGAG |
| | | CCTGCACAGGACCCCCGCCGGCAACTTCGTGACC |
| | | CTGGAGGAGGGCAAGGGCGACCTGGAGGAGTAC |
| | | GGCCAGGACCTGCTGCACACCGTGTTCAAGAACG |
| | | GCAAGGTGACCAAGAGCTACAGCTTCGACGAGAT |
| | | CAGGAAGAACGCCCAGCTGAACATCGAGCTGGAG |
| | | GCCGCCCACCACTGA |
| SEQ ID NO: 71 | Example nucleotide | ATGTACAGGATGCAGCTGCTGAGCTGCATCGCCC |
| | sequence for fusion | TGAGCCTGGCCCTGGTGACCAACAGCTGCCCCCC |
| | protein IgG1 Fc- | CTGCCCCGCCCCCGAGGCCGCCGGCGGCCCCAG |
| | NAMPT 422-491 with IL-2 | CGTGTTCCTGTTCCCCCCCAAGCCCAAGGACACC |
| | signal peptide and | CTGTACATCACCAGGGAGCCCGAGGTGACCTGCG |
| | LALA/YTE mutations | TGGTGGTGGACGTGAGCCACGAGGACCCCGAGG |
| | (underlined and bolded) | TGAAGTTCAACTGGTACGTGGACGGCGTGGAGGT |
| | | GCACAACGCCAAGACCAAGCCCAGGGAGGAGCA |
| | | GTACAACAGCACCTACAGGGTGGTGAGCGTGCTG |
| | | ACCGTGCTGCACCAGGACTGGCTGAACGGCAAGG |
| | | AGTACAAGTGCAAGGTGAGCAACAAGGCCCTGCC |
| | | CGCCCCCATCGAGAAGACCATCAGCAAGGCCAAG |
| | | GGCCAGCCCAGGGAGCCCCAGGTGTACACCCTG |
| | | CCCCCCAGCAGGGACGAGCTGACCAAGAACCAG |
| | | GTGAGCCTGACCTGCCTGGTGAAGGGCTTCTACC |
| | | CCAGCGACATCGCCGTGGAGTGGGAGAGCAACG |
| | | GCCAGCCCGAGAACAACTACAAGACCACCCCCCC |
| | | CGTGCTGGACAGCGACGGCAGCTTCTTCCTGTAC |
| | | AGCAAGCTGACCGTGGACAAGAGCAGGTGGCAGC |
| | | AGGGCAACGTGTTCAGCTGCAGCGTGATGCACGA |
| | | GGCCCTGCACAACCACTACACCCAGAAGAGCCTG |
| | | AGCCTGAGCCCCGGCAAGGGCGGCGGCGGCAGC |
| | | GGCGGCGGCGGCAGCGGCGGCGGCGGCAGCAA |
| | | CAAGAGGAGCAAGAAGGGCAGGCTGAGCCTGCA |
| | | CAGGACCCCCGCCGGCAACTTCGTGACCCTGGAG |
| | | GAGGGCAAGGGCGACCTGGAGGAGTACGGCCAG |
| | | GACCTGCTGCACACCGTGTTCAAGAACGGCAAGG |
| | | TGACCAAGAGCTACAGCTTCGACGAGATCAGGAA |
| | | GAACGCCCAGCTGAACATCGAGCTGGAGGCCGCC |
| | | CACCACTGA |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

Macrophage derived factors as detailed herein are proposed for use in modulating stem cell activity, in directly activating quiescent tissue stem cells and in tissue regeneration. One macrophage derived factor described herein is Nicotinamide phosphoribosyltransferase (NAMPT, also known as visfatin and PBEF (pre-B cell enhancing factor). NAMPT has been identified by the inventors to be upregulated and produced by injury dwelling macrophages.

As described and exemplified herein, the present invention provides a fusion protein comprising:
(i) a NAMPT cytokine finger (cif) polypeptide; and
(ii) an Fc region of an antibody.

Reference to fusion proteins includes variants that activate quiescent tissue stem cells, and their derivatives comprising adaptations suitable for production, and clinical or commercial use, known in the art, such as enhanced tissue delivery or enhanced signalling functionalities. The term includes orthologs and isoforms.

The term “antibody” refers to various antibody structures, including but not limited to monoclonal antibodies, polyclonal antibodies, multispecific antibodies. The term “antibody” may also be used interchangeably with the term “immunoglobulin”.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Particularly, the term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Thus, such recombinant human antibodies have variable and constant regions derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” herein is used to define a C-terminal region of an immunoglobulin heavy chain that contains at least a portion of the constant region. In other words, the Fc region contains at least two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains. The heavy chain constant domains that correspond to the different classes of immunoglobulins are called α, δ, ε, γ, and μ, respectively.

Accordingly, in preferred examples, the fusion proteins of the invention comprise Fc regions which are not capable of homodimerisation. Suitable technology for preventing homodimerisation of Fc regions of antibodies is well known and in some examples may include the use of “knob-into-hole” technology which enables the heterodimerisation of the Fc region of the fusion protein such that the “paired” Fc region does not comprise an NAMPT polypeptide or may comprise an alternative polypeptide fused thereto. Examples of suitable “knob-into-hole” Fc sequences for use in generating monomeric fusion proteins are described in WO 2012/106587 (US20140079689), and also in Ridgeway et al., (1996) Protein Engineering, 9:617-621; incorporated herein by reference.

In another example, the Fc region comprises or consists of a hinge region combined with at least a portion of the heavy chain constant region, preferably the CH2 and CH3 domains of said heavy chain. The hinge region may be derived from an immunoglobulin like molecule or non-immunoglobulin like molecule. In a preferred example, the hinge region comprises or consists of a hinge region from an IgG1, IgG2, IgG3 or IgG4, including a fragment thereof.

In some aspects, the fusion protein of the invention does not exhibit any effector function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibodydependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831).

In another example, an antibody variant may comprise an Fc region with one or more amino acid substitutions which increase the serum half-life of the Fc e.g., substitutions at positions 252, 254 and 256 of the Fc region (EU numbering of residues). For example, the substitutions are M252Y, S254T, T256E.

In preferred examples, the antibody variant comprises an Fc region with L234A, L235A, M252Y, S254T and T256E substitutions.

Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

In some aspects, the Fc region includes mutations to the complement (C1q) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention does not trigger cytotoxicity such as antibody-dependent cellular cytotoxicity (ADCC) or complement dependent cytotoxicity (CDC).

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

Moreover, the herein provided fusion proteins may comprise a linker (or “spacer”). In the context of the present invention, the polypeptide (i.e. NAMPT cif polypeptide) is fused via a linker at the N- or C-terminus to the Fc region. A linker is usually a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided fusion protein may comprise a linker between the NAMPT cif polypeptide and the Fc region of the antibody, such as between the C-terminus of the Fc regions and the N-terminus of the NAMPT cif polypeptide. As another example, the herein provided fusion protein may comprise a linker between the NAMPT cif polypeptide and the Fc region of the antibody, such as between the N-terminus of the Fc regions and the C-terminus of the NAMPT polypeptide. Such linkers have the advantage that they can make it more likely that the different polypeptides of the fusion protein fold independently and behave as expected. Thus, in the context of the present invention the NAMPT polypeptide and the Fc region of an antibody may be comprised in a single-chain multi-functional polypeptide. In some aspects, the fusion protein of the present invention includes a peptide linker. In some aspects, the peptide linker links an NAMPT polypeptide with an Fc region of an antibody. In any embodiment, the peptide linker can include the amino acid sequence Gly-Gly-Ser (GGS), Gly-Gly-Gly-Ser (GGGS, SEQ ID NO: 72) or Gly-Gly-Gly-Gly-Ser (GGGGS, SEQ ID NO: 73). In some aspects, the peptide linker can include the amino acid sequence GGGGS (a linker of 6 amino acids in length) or even longer. The linker may a series of repeating glycine and serine residues (GS) of different lengths, i.e., (GS) n where n is any number from 1 to 15 or more. For example, the linker may be (GS) 3 (i.e., GSGSGS, SEQ ID NO: 74) or longer (GS) 11 or longer. It will be appreciated that n can be any number including 1, 2, 3, 4, 5, 6, 7, 8, 9, 10, 11 or more. Fusion proteins having linkers of such length are included within the scope of the present invention. Preferably n is no more than 3 (ie such that when n equals 3 the linker is GSGSGS, SEQ ID NO: 74). In a preferred embodiment the linker comprises or consists of the sequence shown in SEQ ID NO: 41.

Other exemplary linkers include proline rich linkers such as the sequence PAPAP, APAPA and variations thereof.

In certain embodiments, the NAMPT polypeptide is directly fused to the Fc region of an antibody, such that there is no linker between the two regions of the fusion protein.

The fusion proteins described herein are proposed for use in stimulating wound healing and improving the quality of healing in order to promote full restoration of tissue function i.e., productive tissue repair and regeneration.

The present invention finds particular application in the treatment of, or muscle tissue regeneration in subject having, myopathies where inflammation is particularly undesirable. For example, the invention provides a method of stimulating muscle tissue regeneration in a subject where inflammation is undesirable, the method comprising administering to a muscle an effective amount of a fusion protein, composition or cell as described herein, wherein the fusion protein, composition or cell as described herein, thereby stimulating muscle tissue regeneration in this subject.

Reference to “regeneration” in relation to a muscle is used herein in a broad context and includes the flow on effects on muscle and muscle associated tissue as a direct result of muscle stem cell (also called satellite cell) activation. Thus, regeneration includes muscle wound repair and muscle maintenance, growth, repair, augmentation of the ability of muscle cells to productively proliferate and form functional tissues. The term includes generation of muscle tissue, and repair of an injured muscle, and pertains to the process of muscle regeneration (myogenesis) commencing with activation and proliferation of muscle stem cells, proliferation of myoblasts, early differentiation into myocytes and terminal differentiation into myofibres. In one embodiment, regeneration is associated with minimal fibrosis which allows for establishment of native structures or regenerated tissue having normal or approximating normal biological properties rather than fibrotic or weakened tissue. Muscle functional properties may be determined by standard tests of contractile muscle function, including tests for strength (for example eccentric muscle contraction), power and endurance, as well as physical length and volume. The term also includes growth of muscle tissue in commercial cultures.

In one embodiment, the inflammation that is undesirable is inflammation mediated by TLR activation, preferably TLR4 activation.

In one embodiment, the subject may be diagnosed with an inflammatory myopathy. Exemplary inflammatory myopathies include polymyositis, dermatomyositis, inclusion body myositis, necrotizing autoimmune myopathy. Other inflammatory myopathies, their clinical features and methods of diagnosing them are described in Dalakas, 2015, N Engl J Med 2015; 372:1734-47.

The present invention provides a method of treating an inflammatory myopathy in a subject, the method comprising administering to a muscle of the subject an effective amount of a fusion protein, composition or cell as described herein, thereby treating an inflammatory myopathy. Preferably, the inflammatory myopathy is polymyositis, dermatomyositis, inclusion body myositis or necrotizing autoimmune myopathy.

In one embodiment, the application enables a pharmaceutical or physiologically active regenerative composition comprising one or two or three or four or five of
a fusion protein, nucleic acid, vector, or cell as described herein,
a satellite cell or precursor therefore or progeny thereof
a macrophage or a precursor therefore or progeny thereof
a scaffold or retentive material
a tissue delivery enhancing component.

In a particular embodiment, as described elsewhere herein, the fusion protein in monomeric or dimeric form is modified to make the fusion protein suitable for attachment to a biological carrier or to the extracellular matrix. In addition, the fusion protein is modified to enhance signalling through the CCR5 receptor by addition of moieties that bind co-receptors such as heparin sulphate proteoglycans (such as syndecans).

In one embodiment, a fusion protein, nucleic acid, vector, cell or composition described herein are for use, or for use in manufacturing compositions for use, in stimulating muscle regeneration in vitro, ex vivo or in vitro.

In one embodiment, the compositions described herein are for use or when used in artificial muscle production (such as fish, bird or other non-human animal muscle for direct or indirect consumption). For example, supplementation to growth media enables scalability and more efficient muscle proliferation.

In one embodiment, the compositions described herein are for use or when used in stem cell therapy. Thus, the compositions support expansion in vitro and/or are included in a transplant (or as a pre-treatment) to promote in vivo expansion and tissue integration.

In one embodiment, the present application provides a method of stimulating tissue regeneration, the method comprising administering to an isolated or tissue-resident tissue stem cell or a precursor thereof an effective amount of a composition comprising or encoding a fusion protein described herein, and optionally a component that enhances delivery to the tissue, wherein the fusion protein binds to tissue stem cells or their precursors and stimulates (activates) quiescent tissue stem cell proliferation and tissue regeneration. In one embodiment, the fusion protein comprises a component or moiety that enhances delivery to the target tissue.

In one embodiment, the compositions described herein are for use or when used, or for use in manufacturing compositions for use, in treating a muscular, neuromuscular, or musculoskeletal deficiency, disorder or injury. Muscular, neuromuscular, or musculoskeletal deficiencies, disorders or injuries are known in the art. Deficiencies and disorders are found, for example, and without limitation in sarcopenia, cachexia and the muscular dystrophies, muscle atrophy, muscle pseudo hypertrophy or muscle dystrophy conditions and myopathies. All appropriate formats such as Swiss-style use, method of treatment and/or European-style use claims are encompassed.

The term “isolated cell” as used herein refers to a cell that has been removed from an organism in which it was originally found or a descendant of such a cell. The cell may have been cultured in vitro, e.g., in the presence of other cells. Also, the cell may be destined to later be introduced into a second organism or re-introduced into the organism from which it (or the cell from which it is descended) was isolated.

The term “isolated population of cells” or the like, refers to a population of cells that has been removed and separated from a mixed or heterogeneous population of cells. In some embodiments, an isolated population is a substantially pure population of cells as compared to the heterogeneous population from which the cells were isolated or enriched.

In one embodiment, the application enables a pharmaceutical or physiologically active regenerative composition comprising one or two or three or four or five of:
(i) a fusion protein, nucleic acid, vector, or cell as defined herein,
(ii) a tissue stem cell (such as a satellite cell) or precursor therefore or progeny thereof
(iii) a macrophage or a precursor therefore or progeny thereof
(iv) a scaffold or retentive material
(v) a tissue delivery enhancing component.

In one embodiment, the present application provides cellular compositions comprising one or more of one or more stem cells, stromal cells, pre-satellite cells or satellite cells, pre-macrophages or macrophages or macrophage derived factors as described herein. In one embodiment, multipotent “tissue stem cell” include a pre-muscle cell or any pre-macrophage cells from which these cell may be produced in an essentially native form or modified to express heterologous or autologous factors. Similarly, the term multipotent “tissue stem cell” may include activated progeny of the tissue stem cell.

Tissue stem cells including muscle stem cells can be isolated (for ex vivo or in vitro or in vivo procedures) or induced.

A stem cell can be contacted with a media or composition comprising a fusion protein, nucleic acid, vector, or cell for any amount of time. For example, a stem cell can be contacted with a fusion protein, nucleic acid, vector, or cell for 12 hours, 1 day, 2 days, 3 days, 4 days, 5 days, 6 days, 1 week or more. The stem cell can be induced or stimulated to differentiate into a cell lineage selected from the group consisting of mesoderm, endoderm, ectoderm, neuronal, mesenchymal, and hematopoietic lineage.

In some embodiments, the stem cell is a human stem cell, a multipotent adult stem cell, a pluripotent adult stem cell or an embryonic stem cell.

Human adult stem cells are mitotic and typically one daughter cell remains a stem cell. Adult tissue comprises one or more resident committed progenitor or stem cells that occupy a specific niche in their tissue and actively sense and respond to their local environment. Each tissue typically has its own resident committed stem cell committed to producing progeny that differentiate into a specific range of cell types. Muscle tissue comprises satellite cells to are committed to producing myoblasts. Other well studied stem cells of this type are mesenchymal stem cells (MSC) that produce many different cell types inter alia muscle, cartilage, bone, fat, and haematopoietic stem cells (HSC) that produce all blood cells and the haematopoietic system, and neural stem cells (NSC). All tissue contains resident stem cell populations, including heart, gut and liver. Adult stem cells are typically multipotent which refers to a cell that is able to differentiate into some but not all of the cells derived from all three germ layers. Thus, a multipotent cell is a partially differentiated cell. MSC, for example can be obtained by a number of methods well known in the art. See U.S. Pat. Nos. 5,486,358; 6,387,367; and 7,592,174, and USPN 2003/0211602. MSC may be derived from bone, fat and other tissues where they reside. “Derived” from does not refer to direct derivation and merely indices where they were originally derived.

In one embodiment, the stem cell is a non-embryonic or adult multipotent stem cell.

In one embodiment the stem cell is a HSC or MSC.

Adult stem cell expressing CCR5 can be stimulated to undergo differentiation by exposure to a fusion protein, nucleic acid, vector, or cell described herein. Cells are monitored to changes in expression of for example myogenic regulatory factors known in the art.

Cells may be cultured in standard media or specifically defined media.

Cell expression may be modified by techniques know in the art.

An induced or partially induced pluripotent stem cell is a convenient source of stem cells. These are derivable from a differentiated adult cell, such as human foreskin cells.

Human iPS cells can be generated by introducing specific sets of reprogramming factors into a non-pluripotent cell which can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. For example, the reprograming factors can be introduced into the cells using one or more plasmids, lentiviral vectors, or retroviral vectors. In some cases, the vectors integrate into the genome and can be removed after reprogramming is complete. In some cases, the vectors do not integrate (e.g., those based on a positive-strand, single-stranded RNA species derived from non-infectious (non-packaging) self-replicating Venezuelan equine encephalitis (VEE) virus, Simplicon RNA Reprogramming Kit, Millipore, SCR549 and SCR550). The Simplicon RNA replicon is a synthetic in vitro transcribed RNA expressing all four reprogramming factors (OKG-iG; Oct4, Klf4, Sox2, and Glis1) in a polycystronic transcript that is able to self-replicate for a limited number of cell divisions. Human induced pluripotent stem cells produced using the Simplicon kit are referred to as “integration-free” and “footprint-free.” Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells express alkaline phosphatase, SOX-2, OCT-4, Nanog and Tra-1-60 markers.

The terms “naive” and “primed” identify different pluripotency states of human iPS cells. Characteristics of naive and primed iPS cells are described in the art. Naive human iPS cells exhibit a pluripotency state similar to that of ES cells of the inner cell mass of a pre-implantation embryo. Such naive cells are not primed for lineage specification and commitment. Female naive iPS cells are characterized by two active X chromosomes. In culture, self-renewal of naive human iPS cells is dependent on leukemia inhibitory factor (LIF) and other inhibitors. Cultured naive human iPS cells display a clonal morphology characterized by rounded dome-shaped colonies and a lack of apico-basal polarity. Cultured naive cells can further display one or more pluripotency makers as described elsewhere herein. Under appropriate conditions, the doubling time of naive human iPS cells in culture can be between 16 and 24 hours.

Primed human iPSC express a pluripotency state similar to that of post-implantation epiblast cells. Such cells are primed for lineage specification and commitment. Female primed iPSCs are characterized by one active X chromosome and one inactive X chromosome. In culture, self-renewal of primed human iPSCs is dependent on factors such as fibroblast growth factor (FGF) and activin. Cultured primed human iPSCs display a clonal morphology characterized by an epithelial monolayer and display apico-basal polarity. Under appropriate conditions, the doubling time of primed human iPSCs in culture can be 24 hours or more depending upon the level from the adult cells from which they were derived.

Embryonic stem cells (ESC) are characteristically pluripotent i.e., they have the capacity, under different conditions, to differentiate to cell types characteristic of all three germ cell layers (endoderm, mesoderm and ectoderm). Pluripotent cells are characterized primarily by their ability to differentiate to all three germ layers. In some embodiments, a pluripotent cell is an undifferentiated cell. Pluripotent cells also have the potential to divide in vitro for more than one year or more than 30 passages.

ESC are typically the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see U.S. Pat. Nos. 5,945,577, 5,994,619, 6,235,970). Exemplary distinguishing embryonic stem cell characteristics include, without limitation, gene expression profile, proliferative capacity, differentiation capacity, karyotype, responsiveness to particular culture conditions.

In one embodiment the stem cell is adult.

In one embodiment the stem cells are autologous or heterologous to the subject.

In one embodiment, the stem cell is mammalian or human.

Macrophages and stem cells including satellite cells may be prepared using art recognised methods and as described herein and include the use of iPSC and optionally gene editing procedures.

One embodiment isolated macrophages or stem-cell derived macrophages are modified to express a polypeptide or fusion protein described herein. Generally, M2 type macrophages are selected or provided.

In one embodiment stem cells are contacted with a fusion protein, nucleic acid, vector, or cell in vitro, ex vivo or in vivo as described herein to induce activation and proliferation. Stem cells treated in vitro or ex vivo may be introduced into a wound site to effect repair or administered systemically to effect regeneration of damaged tissue or to treat or improve muscle related conditions as described herein.

A fusion protein, nucleic acid, vector, or cell expressing said fusion protein may be administered in the form of functionalized hydrogels either alone or together with cells for transplantation. Such hydrogels or similar biomaterials or scaffolds provide enhanced transplantation efficiency at the wound site.

Hydrogels may be ECM based such as fibrin based. Alternatively, hydrogels may be non-ECM based such as acrylamide based using RAFT technology (see Chiefari et al Macromol. 31:5559-5526, 1998 and Fairbanks et al Advanced Drug Delivery Reviews 91:141-152, 2015). Suitable materials regulate release kinetics, and have desired mechanical and physical properties for tissue regeneration as known in the art.

In one embodiment, satellite cells are encapsulated in CCR5 functionalised hydrogels or other biomaterials.

Kits comprising the cellular compositions and/or agents described herein are also provided. Kits suitable for muscle repair or regeneration are specifically contemplated. The fusion protein, nucleic acid, vector, or cell can be pre-formulated for administration or ingredients for formulation can be provided with the kit. The fusion protein, nucleic acid, vector, or cell is for example formulated in a hydrogel or other supporting vehicle for topical application. The fusion protein, nucleic acid, vector, or cell may be, for example, lyophilised or liquid.

The terms “protein,” “polypeptide,” and “peptide,” used interchangeably herein, include polymeric forms of amino acids of any length unless that length is defined, including coded and non-coded amino acids and chemically or biochemically modified or derivatized amino acids. The terms also include polymers that have been modified, such as polypeptides having modified peptide backbones.

Proteins are said to have an “N-terminus” be “N-terminal” and to have a “C-terminus” or be “C-terminal.” The term “N-terminus” relates to the start of a protein or polypeptide, terminated by an amino acid with a free amine group (—NH2). The term “C-terminus” relates to the end of an amino acid chain (protein or polypeptide), which is in nature terminated by a free carboxyl group (—COOH). In the present application reference to C-terminal and N-terminal fragments broadly describe the region of the full length molecule from which the elected part is derived and it excludes a full length or a native molecule. A C-terminal fragments does not have to but may include all the C-terminal amino acids and N-terminal fragments do not have to but may include all the N-terminal amino acids.

The application discloses and enables the use of a range of fusion proteins, nucleic acids, vectors, or cells based upon the initial findings described in the examples.

A number of peptide modifications are known in the art to stabilise peptides against serum proteases or to promote intracellular positioning and these are encompassed. Some such modified peptides can be expressed from nucleic acids in a cell, others are manufactured synthetically. Similarly, where it is desirable to target the fusion protein described herein to one or more specific cell types, this may be achieved either by ex vivo manipulation of target cells, or incorporation of targeting moieties able to bind specifically to target cells or tissues such as ECM binding moieties, as known in the art.

A “conservative amino acid substitution” is one in which the naturally or non-naturally occurring amino acid residue is replaced with a naturally or non-naturally occurring amino acid residue having a similar side chain. Families of amino acid residues having similar side chains have been defined in the art. These families include amino acids with basic side chains (e.g., Lys, Arg, His), acidic side chains (e.g., Asp, Glu), uncharged polar side chains (e.g., Gly, Asn, Gln, Ser, Thr, Tyr, Cys), nonpolar side chains (e.g., Ala, Val, Leu, Ile, Pro, Phe, Met, Trp), beta-branched side chains (e.g., Thr, Val, Ile) and aromatic side chains (e.g., Phe, Trp, His). Thus, a predicted nonessential amino acid residue in a fusion protein as described herein, for example, may be replaced with another amino acid residue from the same side chain family. Other examples of acceptable substitutions are substitutions based on isosteric considerations (e.g. norleucine for methionine) or other properties (e.g. 2-thienylalanine for phenylalanine). A full amino acid sub-classification is set out in Table 2 and exemplary substitutions are set out in Table 3.

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

TABLE 3
Exemplary and Preferred Amino Acid Substitutions

| Original residue | Exemplary substitutions | Preferred substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, | Leu |
| | Norleu | |
| Leu | Norleu, Ile, Val, Met, Ala, | Ile |
| | Phe | |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, | Leu |
| | Norleu | |

In some embodiments Trp residues are substituted.

A fusion protein described herein may comprise modifications known to modify the pharmacokinetic features of peptides, such as by increasing protease resistance in vivo.

In one embodiment, the peptide comprises one or more of a linker or spacer such as GGS or repeats of GGS and variants known in the art), a modified or non-natural or non-proteogenic amino acid, a modified side-chain, a modified backbone, terminal modified groups or comprises a modified spatial constraint or is a D-retro-inverso peptide. In one embodiment, the peptide is a pseudopeptide, peptoid, azapeptide, cyclized, stapled, ether or lactam peptide or comprises a spatial constraint.

In one embodiment, the fusion protein described herein is conjugated or otherwise attached/bound/expressed with as appropriate to a lipid, carbohydrate, polymer, protein, nanoparticle, peptide, proteoglycan, antibody or fragment or antigen binding form thereof, aptamer, or nucleic acid.

In one embodiment, the fusion protein described herein specifically binds to muscle cells or muscle cell tissue or associated structures eg, ECM.

In one embodiment, the fusion protein described herein includes physiologically or pharmaceutically acceptable salts, hydrates, stereoisomers, and pro-drugs.

In one embodiment, non-essential amino acids may be altered. Reference to “non-essential” amino acid residue means a residue that can be altered from the wild-type sequence of a polypeptide without abolishing or substantially altering its ability to bind to an endogenous or heterologous CCR5.

In one embodiment, the fusion protein described herein comprises or encodes an amino acid sequence having 1, 2, 3, 4, 5 or 6 conservative (for example those outlined in Table 3 above) or non-conservative amino acid substitution, deletion or addition to the above sequences but retains CCR5 interacting activity.

In one embodiment, the invention also provides a nucleic acid molecule from which the fusion protein described herein is expressible. Nucleotide sequences encoding fusion proteins described herein are herein disclosed.

In one embodiment, the nucleic acid molecule is an RNA or DNA or RNA: DNA or a chemically modified form thereof.

In one embodiment, a proportion of at least one type of nucleotide (e.g, cysteine and/or uracil), is chemically modified to increase its stability in vivo.

In one embodiment, the nucleic acid is in the form of a viral or non-viral vector.

In one embodiment, the fusion protein, nucleic acid, vector, cell or composition described herein is administered to cells ex vivo. The present invention encompasses the use of genetically modified cell depots (e.g. genetically modified macrophage, etc).

In one embodiment, the fusion protein further comprises an antibody or antibody fragment that targets the agent specifically to target cells, such as muscle stem cells.

In one embodiment, the present application provides a pharmaceutical or physiological composition comprising a fusion protein, nucleic acid, vector or cell as defined herein above.

The application enables a method of treating a muscle injury or a person with a diminished or suboptimal ability to repair or regenerate muscle, comprising administering to the subject an effective amount of a composition comprising a fusion protein, nucleic acid, vector or cell, or composition comprising a fusion protein, nucleic acid, vector or cell, sufficient to stimulate muscle stem cell proliferation and muscle regeneration.

Compositions include physiologically or pharmacologically or pharmaceutically acceptable vehicles that are not biologically or otherwise undesirable.

Pharmacologically acceptable salts, esters, pro-drugs, or derivatives of a compound described here is a salt, ester, pro-drug, or derivative that is not biologically or otherwise undesirable.

In some embodiments, the fusion protein is modified. Fusion protein activity are tolerant to additional moieties, flanking residues and substitutions within the defined boundaries. Similarly backbone modifications and replacements, side-chain modifications and N and C-terminal modifications are conventional in the art. Generally, the modification is to enhance stability or pharmacological profile, targeting/delivery. For example, peptide cyclisation or stapling is conventional for enhancing peptide stability. In another embodiment, peptides or agents are in the form of micro or nano-particles or bubbles, gels, liposomes, conjugates or fusion proteins comprising moieties adapted for stability, delivery or specificity to the target tissue.

In one embodiment, fusion proteins or their encoding nucleic acids where appropriate are assembled in liposomes, hydrogels, emulsions, viral vectors, viral-like particles or virosomes.

In one embodiment, specific binding moieties such as antibody or antibody fragments or mimics are used to target fusion proteins to the muscle environment.

In one embodiment, fusion proteins are delivered through biological synthesis in vivo such as via delivery of mRNA, gene editing such as CRISPR components, or bacteria or cells.

Compositions generally comprise a fusion protein, peptidomimetic or an encoding nucleic acid where appropriate, and a pharmaceutically acceptable carrier and/or diluent. In one embodiment, the carrier may be a nanocarrier.

In one embodiment, the fusion proteins of the present disclosure are not naturally occurring molecules, but instead are modified forms of naturally occurring molecules which do not possess certain features or functions of the naturally occurring full length molecules. For example, NAMPT enzymatic activity may be absent.

In another embodiment, the fusion protein described herein is constrained by means of a linker which is covalently bound to at least two amino acids in the peptide. Various cyclisation strategies are known in the art to increase stability and cellular permeability.

In some embodiments, the fusion protein described herein is delivered in the form of nucleic acid molecules encoding same or pro-drugs thereof or vectors comprising nucleic acid molecules encoding same or pro-drugs thereof. In one embodiment, the nucleic acid is mRNA. The fusion protein described herein may bind the surface of the muscle stem cell or function internally to stimulate signalling and proliferation.

In another embodiment, the fusion protein, nucleic acid, vector or cell described herein is combined, in addition to other pharmaceutically acceptable carriers, with amphipathic agents such as lipids which exist in aggregates as micelles, insoluble monolayers, liquid crystals or lamellar layers in aqueous solution.

In one embodiment, the disclosure enables a composition comprising a fusion protein as described herein which interacts with endogenous CCR5 proteins for use as a medicament or for use in therapy.

In another aspect, the present disclosure enables a composition for stimulating muscle stem cell proliferation comprising a fusion protein as described herein or a nucleic acid molecule from which the peptide is expressible.

In one embodiment, the subject composition is co-administered with a second physiologically active, therapeutic or prophylactic or regenerative agent. Illustrative cytokines include without limitation one or more of IGF-1, TGF-β, GDF-5, bFGF, PDGF-b3, IL-4.

In another aspect, the present disclosure provides for the use of the fusion protein, nucleic acid, vector or cell as described herein in the manufacture of a medicament for stimulating muscle regeneration or in stem cell therapy.

Reference to “amino acid” includes naturally occurring amino acids or non-naturally occurring amino acids.

Peptide compounds are generally and conventionally modifiable by addition of moieties, flanking peptide residues, and substitutions within understood parameters. Peptides can furthermore comprise routine modified backbones, side chains, peptide bond replacements, and terminal modifications using standard peptide chemistries.

The amino acids incorporated into the amino acid sequence described herein may be L-amino acids, D-amino acids, L-β-homo amino acids, D-β-homo amino acids or N-methylated amino acids, sugar amino acids, and/or mixtures thereof. Non-natural amino acids may not be recognised by proteases and may therefore alter the half-life. In one embodiment, the D-retro inversion sequence is employed.

Non-naturally occurring amino acids include chemical analogues of a corresponding naturally occurring amino acid. Examples of unnatural amino acids and derivatives include, but are not limited to, 4-amino butyric acid, 6-aminohexanoic acid, 4-amino-3-hydroxy-5-phenylpentanoic acid, 4-amino-3-hydroxy-6-methylheptanoic acid, t-butylglycine, nor leucine, norvaline, phenylglycine, ornithine, sarcosine, 2-thienyl alanine and/or D-isomers of amino acids.

The term “amino acid analog” refers to a molecule which is structurally similar to a naturally occurring amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group or for the substitution of the amino or carboxy group by a similarly reactive group (e.g., substitution of the primary amine with a secondary or tertiary amine, or substitution or the carboxy group with an ester).

Linkers or spacers may be amino acids or nucleic acids or other atomic structures known in the art, typically between 2 and 10 amino acids or nucleotides in length. Spacers should be flexible enough to allow correct orientation of a fusion protein as described herein, such as those including nanoparticles, antibody fragments, liposomes, cell penetrating and/or intracellular delivery moieties. One form of spacer is the hinge region from IgG suitable for use when the construct comprises an antigen binding moiety for cellular targeting.

In one embodiment, a fusion protein described herein is conjugated or otherwise associated (covalent or non-covalent attachment) with a delivery agent. In one embodiment the delivery agent delivers the peptide to tissue, a target cell or cell population.

Derivatives of fusion proteins described herein include biologically active fragments thereof as described herein comprising the structures described or orthologs.

Derivatives also include molecules having a percent amino acid or polynucleotide sequence identity over a window of comparison after optimal alignment. In one embodiment the percentage identity is at least 80%-99% including any number in between 80 and 99.

Suitable assays for the biological activity of fusion proteins are known to the skilled addressee and are described in the specification.

In some embodiment, markers of fusion protein activity include upregulation of satellite cell signalling (eg, MAPK), stem cell and myoblast proliferation and differentiation.

In one embodiment, the fusion protein is modified with a moiety which is not a naturally occurring amino acid residue. The moiety may be selected from the group consisting of a detectable label, a non-naturally occurring amino acid as described herein, a reactive group, a fatty acid, cholesterol, a lipid, a bioactive carbohydrate, a nanoparticle, a small molecule drug, and a polynucleotide. In one particular embodiment, the moiety is a detectable tag label. In one example the detectable label is selected from the group consisting of a fluorophore, a fluorogenic substrate, a luminogenic substrate, and a biotin. Art recognised tags or labels include affinity agents and moieties for detection include fluorescent and luminescent compounds, metals, dyes. Other useful moieties include affinity tags, biotin, lectins, chelators, lanthanides, fluorescent dyes, FRET acceptor/donors.

In one embodiment, the fusion protein which may comprise a detectable label, is accompanied in a kit with a modified control version of the fusion protein wherein the conserved residues of the fusion protein are substituted with for example alanine. Kits comprising the agents are proposed for sale and may be used for screening purposes or therapeutic purposes.

Peptides of this type may be obtained through the application of recombinant nucleic acid techniques as, for example, described in Sambrook et al. MOLECULAR CLONING. A LABORATORY MANUAL (Cold Spring Harbour Press, 1989), in particular Sections 16 and 17; Ausubel et al CURRENT PROTOCOLS IN MOLECULAR BIOLOGY (John Wiley & Sons, Inc. 1994-1998), in particular Chapters 10 and 16; and Coligan et al. CURRENT PROTOCOLS IN PROTEIN SCIENCE (John Wiley & Sons, Inc. 1995-1997), in particular Chapters 1, 5 and 6.

Alternatively, peptides of this type may be synthesised using conventional liquid or increasingly solid phase synthesis techniques. For example, initial reference may be made to solution synthesis or solid phase synthesis as described, for example, by Atherton and Sheppard in SOLID PHASE PEPTIDE SYNTHESIS: A PRACTICAL APPROACH (IRL Press at Oxford University, Oxford, England, 1989), see particularly Chapter 9, or by Roberge et al. (1995 Science 269:202).

Azapeptide synthesis was previously hampered by tedious solution-phase synthetic routes for selective hydrazine functionalization. Recently, the submonomer procedure for azapeptide synthesis, has enabled addition of diverse side chains onto a common semicarbazone intermediate, providing a means to construct azapeptide libraries by solution- and solid-phase chemistry. In brief, aza residues are introduced into the peptide chain using the submonomer strategy by semicarbazone incorporation, deprotonation, N-alkylation, and orthogonal deprotection. Amino acylation of the resulting semicarbazide and elongation gives the desired azapeptide. Furthermore, a number of chemical transformations have taken advantage of the orthogonal chemistry of semicarbazone residues (e.g., Michael additions and N-arylations). In addition, oxidation of aza-glycine residues has afforded azopeptides that react in pericyclic reactions (e.g., Diels-Alder and Alder-ene chemistry). The bulk of these transformations of aza-glycine residues have been developed by the Lubell laboratory, which has applied such chemistry in the synthesis of ligands with promising biological activity for treating diseases such as cancer and age-related macular degeneration. Azapeptide analogues of growth hormone-releasing peptide-6 (His-d-Trp-Ala-Trp-d-Phe-Lys-NH2, GHRP-6) have for example been pursued as ligands of the cluster of differentiation 36 receptor (CD36) and show promising activity for the development of treatments for angiogenesis-related diseases, such as age-related macular degeneration, as well as for atherosclerosis. Azapeptides have also been employed to make a series of conformationally constrained second mitochondria-derived activator of caspase (Smac) mimetics that exhibit promising apoptosis-inducing activity in cancer cells. The synthesis of cyclic azapeptide derivatives was used to make an aza scan to study the conformation-activity relationships of the anticancer agent cilengitide, cyclo(RGDf-N (Me) V), and its parent counterpart cyclo(RGDfV), which exhibit potency against human tumor metastasis and tumor-induced angiogenesis. Innovations in the synthesis and application of azapeptides are described in Acc Chem Res. 2017 Jul. 18; 50 (7): 1541-1556.

Alternatively, peptides can be produced by digestion of an adaptor polypeptide with proteinases such as endoLys-C, endoArg-C, endoGlu-C and Staphylococcus V8-protease. The digested fragments can be purified by, for example, high performance liquid chromatographic (HPLC) techniques. Measures that may be taken to optimize pharmacodynamics parameters of peptides and peptide analogs are described by Werle M. et al (2006) Strategies to improve plasma half-life time of peptide and protein drugs amino Acids 30 (4): 351-367; and Di L (2014) Strategic approaches to optimising peptide ADME properties AAPS J 1-10.

The fusion protein may be stabilised for example via nanoparticles, liposomes, micelles or for example PEG as known in the art. Methods to form liposomes are described in: Prescott, Ed. Methods in Cell Biology, Volume XIV, Academic Press, New York, N.Y. (1976), p. 33 et seq., the contents of which is incorporated herein by reference. Polymer nanoparticles ideally use surfactants that are not toxic or physically adsorbed to the nanoparticle. In one aspect, biodegradable surfmers are used. For example, biodegradable, poly(ethylene glycol) (PEG)ylated N-(2-hydroxypropyl) methacrylamide (HPMA) based surfmers are synthesized and used to stabilize lipophilic NPs. In particular, the NP core is made from a macromonomer comprising a poly(lactic acid) (PLA) chain functionalized with HPMA double bond. The nanoparticle forming polymer chains are then constituted by a uniform poly (HPMA) backbone that is biocompatible and water soluble and hydrolysable PEG and PLA pendants assuring the complete degradability of the polymer. The stability provided by the synthesized surfmers is studied in the cases of both emulsion free radical polymerization and solution free radical polymerization followed by the flash nanoprecipitation of the obtained amphiphilic copolymers.

Other stabilising or heterologous moieties include NMEG, ECM binding, syndecan binding albumin, albumin binding proteins, immunoglobulin Fc domain.

In some embodiments, nanoparticles comprising the fusion protein, nucleic acid, vector or cell can be further modified by the conjugation of tissue type specific binding agents, antibodies or fragments thereof known in the art.

Fusion proteins described herein may be administered in a range of retention enhancing compositions known in the art, such as gels, foams, glues, hydrogels, patches, and films, and the like.

Oral and injectable solution solubilizing excipients include water-soluble organic solvents (polyethylene glycol 300, polyethylene glycol 400, ethanol, propylene glycol, glycerin, N-methyl-2-pyrrolidone, dimethylacetamide, and dimethylsulfoxide), non-ionic surfactants (Cremophor EL, Cremophor RH 40, Cremophor RH 60, d-α-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750), water-insoluble lipids (castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil), organic liquids/semi-solids (beeswax, d-α-tocopherol, oleic acid, medium-chain mono- and diglycerides), various cyclodextrins (α-cyclodextrin, β-cyclodextrin, hydroxypropyl-β-cyclodextrin, and sulfobutylether-β-cyclodextrin), and phospholipids (hydrogenated soy phosphatidylcholine, distearoylphosphatidylglycerol, I-α-dimyristoylphosphatidylcholine, I-α-dimyristoylphosphatidylglycerol). The chemical techniques to solubilize agents for oral and injection administration include pH adjustment, cosolvents, complexation, microemulsions, self-emulsifying drug delivery systems, micelles, liposomes, and emulsions.

### Constructs/Vectors

A construct or vector for expressing a fusion protein described herein from a recipient cell can comprise one or more DNA regions comprising a promoter operably linked to a nucleotide sequence encoding the peptide. The promoter can be inducible or constitutive. Examples of suitable constitutive promoters include, e.g., an immediate early cytomegalovirus (CMV) promoter, an Elongation Growth Factor-Ia (EF-Ia) gene promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Examples of inducible promoters include, but are not limited to a metallothionine promoter, a glucocorticoid promoter, a progesterone promoter, and a tetracycline promoter.

The expression constructs may be generated by any suitable method including recombinant or synthetic techniques, utilizing a range of vectors known and available in the art such as plasmids, bacteriophage, baculovirus, mammalian virus, artificial chromosomes, among others. The expression constructs can be circular or linear, and should be suitable for replication and integration into eukaryotes. Viruses, which are useful as vectors include, but are not limited to, retroviruses, adenoviruses, adeno-associated viruses, herpes viruses and lentiviruses. A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to the subject stem cells. A number of retroviral systems are known in the art.

In a specific embodiment of the present invention, where the fusion protein is provided as a fusion protein encoding the peptide, the nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide or other intracellular targeting moiety. Alternatively, a nucleic acid can be introduced intracellularly and incorporated within host cell DNA for expression.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” may be used and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A nucleic acid molecule as described herein may in any form such as DNA or RNA, including in vitro transcribed RNA or synthetic RNA. Nucleic acids include genomic DNA, cDNA, RNA, recombinantly produced and chemically synthesized molecules and modified forms thereof. A nucleic acid molecule may be single stranded or double stranded and linear or closed covalently to form a circle. The RNA may be modified by stabilizing sequences, capping, and polyadenylation. RNA or DNA and may be delivered as plasmids to express the peptide. RNA-based approaches are routinely available.

The term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

An optimised mRNA based composition could comprise a 5′ and 3′ non translated region (5′-UTR, 3′-UTR) that optimises translation efficiency and intracellular stability as known in the art. In one embodiment, removal of uncapped 5′-triphosphates can be achieved by treating RNA with a phosphatase. RNA may have modified ribonucleotides in order to increase its stability and/or decrease cytotoxicity. For example, in one embodiment, in the RNA, 5-methylcytidine is substituted partially or completely, for cytidine. In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap. The term “5′-cap” includes a 5′-cap analog that resembles the RNA cap structure and is modified to possess the ability to stabilize RNA and/or enhance translation of RNA. Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

A further modification of RNA may be an extension or truncation of the naturally occurring poly(A) tail or an alteration of the 5′- or 3 ‘-untranslated regions (UTR) such as introduction of a UTR which is not related to the coding region of said RNA, for example, the exchange of the existing 3’-UTR with or the insertion of one or more, preferably two copies of a 3′-UTR derived from a globin gene, such as alpha2-globin, alphal-globin, beta-globin. RNA having an unmasked poly-A sequence is translated more efficiently than RNA having a masked poly-A sequence. In order to increase stability and/or expression of the RNA it may be modified so as to be present in conjunction with a poly-A sequence, preferably having a length of 10 to 500, more preferably 30 to 300, even more preferably 65 to 200 and especially 100 to 150 adenosine residues. In order to increase expression of the RNA it may be modified within the coding region so as to increase the GC-content to increase mRNA stability and to perform a codon optimization and, thus, enhance translation in cells. Modified mRNA may be synthesised enzymatically and packaged into nanoparticles such as lipid nanoparticles and administered, for example intramuscularly.

The nucleic acid molecule can be entrapped in microcapsules prepared, for example, by coacervation techniques or by interfacial polymerization, in colloidal drug delivery systems (e.g., liposomes, microspheres, microemulsions, nanoparticles and nanocapsules), or in macroemulsions. Such techniques are known in the art and disclosed in Remington, the Science and Practice of Pharmacy, 20th Edition, Remington, J., ed. (2000). Targeted delivery of agents to particular cell subsets can enhance the therapeutic index. Antibody targeted agents that bind to cells comprising an antigen recognized by the antibody or binding fragments thereof. This include for example maleimide functionalized PEG-PLGA polymeric nanoparticles, or simply combining a fusion protein described herein in a composition comprising a delivery moiety or shuttle agent.

Ex vivo approaches contemplate the administration of gene editing such as CRISPR components to modify cells to contain or express a fusion protein as described herein.

### Administration

In accordance with this disclosure, the fusion proteins, nucleic acids, vectors, or cells as described herein, or compositions comprising or encoding the same, can be administered to patients for wound healing or to delay, maintain, or regenerate muscle in various conditions associated with muscle loss or diminished ability to regenerate functionally.

The fusion proteins, nucleic acids, vectors, or cells as described herein, or compositions comprising or encoding the same, may be delivered by injection, by topical or mucosal application, by inhalation or via oral route including modified release modes, over periods of time and in amounts which are effective to stimulate muscle regeneration levels in a subject. Administration may be topical or systemic (e.g., parenteral via for example intravenous, intraperitoneal, intradermal, sub cutaneous or intramuscular routes) or targeted. In one embodiment, administration of CCR5-interacting agent is systemic or directly to a wound. Sub cutaneous or intramuscular routes may be directly to an affected muscle tissue.

A fusion protein, nucleic acid, vector, or cell as described herein, or compositions comprising or encoding the same, can be formulated in the form of ointments, creams, patches, powders, or other formulations suitable for topical formulations. Small molecular weight polypeptide or fusion protein formulations can deliver the agent from skin to deeper muscle tissue. Accordingly, such formulations may comprise one or more agents that enhance penetration of active ingredient through skin. For topical applications, A fusion protein, nucleic acid, vector, or cell as described herein, or compositions comprising or encoding the same, can be included in wound dressings and/or skin coating compositions.

The amount of the agent to be administered may be determined by standard clinical techniques by those of average skill within the art. In addition, in vitro assays may optionally be employed to help identify optimal dosage ranges. The precise dose to be employed will also depend on the nature of the agent and other clinical factors (such as the condition of the subject their weight, age, other conditions, the route of administration and type of composition (cellular, scaffolded, hydrogel baes or oral formulations). The precise dosage to be therapeutically or prophylactically effective and non-detrimental can be determined by those skilled in the art. Pharmaceutical compositions are conveniently prepared according to conventional pharmaceutical compounding techniques. See, for example, Remington, the Science and Practice of Pharmacy, 20th Edition, Remington, J., ed. (2000) and later editions.

Reference to an effective amount includes a therapeutically or physiologically or regeneratively effective amount. A “therapeutically-effective amount” as used herein means that amount of the composition comprising chemokine receptor agonist activity which is effective for producing some desired therapeutic effect in at least a sub-population of cells in an animal at a reasonable benefit/risk ratio applicable to any medical treatment. For example, an amount of a polypeptide or fusion protein administered to a subject that is sufficient to produce a statistically significant, measurable muscle repair or regeneration. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. Routes of administration suitable for the instant compositions with vary depending upon its format and include both local and systemic administration. Generally, local administration results in more of the fusion proteins, nucleic acids, vectors, or cells as described herein, or compositions comprising or encoding the same, being delivered to a specific location as compared to the entire body of the subject, whereas, systemic administration results in delivery to essentially the entire body of the subject. One method of local administration is by intramuscular injection.

In accordance with the present invention, the term “administering” also include transplantation of a cell into a subject. As used herein, the term “transplantation” refers to the process of implanting or transferring at least one cell into a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). Skilled artisan is well aware of methods for implanting or transplantation of stem cells for muscle repair and regeneration, which are amenable to the present invention. See for example, U.S. Pat. No. 7,592,174 and U.S. Pat. Pub. No. 2005/0249731, content of both of which is herein incorporated by reference.

As described herein regeneration of muscle tissue by the present methods may be associated with minimal fibrosis. Specifically, the methods and agents described herein may reduce and/or inhibit formation of scar-like tissue in the damaged or non-regenerating or atrophying muscle tissue. Accordingly, in some embodiments, formation of scar-like tissue formation in the damaged muscle tissue is reduced by at least 5%, at least 10%, at least 20%, at least 30%, at least 40%, at least 50%, at least 60%, at least 70%, at least 80%, at least 90%, at least 95%, or 100% relative to a control without the present agents. Adipose deposition may be similarly reduced.

However, suitable dosage ranges for intravenous administration of a fusion protein, nucleic acid, vector, or cell as described herein, or compositions comprising or encoding the same, are generally about 1.25-5 micrograms of active compound per kilogram (Kg) body weight. Suitable dosage ranges for intranasal administration are generally about 0.01 μg/kg body weight to 1 mg/kg body weight. Effective doses may be extrapolated from dose-response curves derived from in vitro or animal model test systems. Suppositories generally contain active ingredient in the range of 0.5% to 10% by weight; oral compositions preferably contain 10% to 95% active ingredient.

By “derivative” is meant an agent or active that has been derived from the fusion protein by modification of the amino acid sequence, or, for example by conjugation or complexing or expression (eg, as a fusion protein) with other chemical moieties or by post-translational modification techniques as would be understood in the art. The term “derivative” also includes within its scope alterations that have been made to a parent sequence including additions, or deletions that provide for functionally equivalent or functionally enhanced molecules.

By “isolated” is meant material that is substantially or essentially free from components that normally accompany it in its native state.

The term “subject,” includes patient, and refers to any subject of medical or veterinary interest. Subjects may be a vertebrate subject, such as mammalian subject (e.g, bovines, pigs, dogs, cats, equine, lama, camelids, etc.), non-mammals, reptiles, birds, fish. The subject includes a human, for whom prophylaxis or therapy is desired. The subject may be in need of prophylaxis or treatment for a cancer, wound care, sarcopenia or other pathology, disease, disorder or condition associated with tissue degeneration.

The term “polynucleotide” or “nucleic acid” as used herein designates mRNA, RNA, CRNA, cDNA or DNA. The term typically refers to oligonucleotides greater than 30 nucleotides in length.

The term sequence “identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base {e.g., A, T, C, G, U) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” may be understood to mean the “match percentage” calculated by the DNASIS computer program (Version 2.5 for Windows; available from Hitachi Software Engineering Co., Ltd., South San Francisco, California, USA) using standard defaults as used in the reference manual accompanying the software. Amino acid sequence identity may also be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term sequence “similarity” refers to the percentage number of amino acids that are identical or constitute conservative amino acid substitutions as defined in Table 3 above. Similarity may be determined using sequence comparison programs such as GAP (Deveraux et al, 1984 Nucleic Acids Research 12:387-395). In this way, sequences of a similar or substantially different length to those cited herein might be compared by insertion of gaps into the alignment, such gaps being determined, for example, by the comparison algorithm used by GAP. Methods involving conventional molecular biology techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., (2001); and Current Protocols in Molecular Biology, ed. Ausubel et al., Greene Publishing and Wiley-Interscience, New York, (1992) (with periodic updates). Immunology techniques are generally known in the art and are described in detail in methodology treatises such as Current Protocols in Immunology, ed. Coligan et al., Greene Publishing and Wiley-Interscience, New York, (1992) (with periodic updates); Advances in Immunology, volume 93, ed. Frederick W. Alt, Academic Press, Burlington, Mass., (2007); Making and Using Antibodies: A Practical Handbook, eds. Gary C. Howard and Matthew R. Kaser, CRC Press, Boca Raton, Fl, (2006); Medical Immunology, 6th ed., edited by Gabriel Virella, Informa Healthcare Press, London, England, (2007); and Harlow and Lane ANTIBODIES: A Laboratory Manual, Second edition Cold Spring Harbor Laboratory Press, Cold Spring Harbor, N.Y., (2014). Conventional methods of gene transfer and gene therapy may also be adapted for use in the present invention. See, e.g., Gene Therapy: Principles and Applications, ed. T. Blankenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010. Amino Acids. 2018 January; 50 (1): 39-68. doi: 10.1007/s00726-017-2516-0. Epub 2017 Nov. 28.

It will be understood that the invention disclosed and defined in this specification extends to all alternative combinations of two or more of the individual features mentioned or evident from the text or drawings. All of these different combinations constitute various alternative aspects of the invention.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Materials and Methods

### Mouse Volumetric Muscle Loss Injury and Repair Assessment

Injury: male C57BL/6J mice aged between 10-12 weeks were anesthetised and shaved on the hind left leg. A unilateral incision measuring approximately 1 cm was made exposing the underlying fascia. The left hind limb was extended and exteriorised via the incision site by retracting the surrounding tissue. A 3×4 mm full thickness segment of the rectus femoris muscle was removed. Directly after, the injury site was filled with fibrin hydrogel with or without NAMPT (0.5 μg), NAMPT₄₂₂₋₄₉₁ (0.096 μg), IgG1Fc NAMPT₄₂₂₋₄₉₁ (0.32 μg) (hydrogel components; 40 μl, 8 mg/ml human fibrinogen (FIB3, Enzyme Research Laboratories), 4 U/ml bovine thrombin (T4648, Sigma), 5 mM CaCl2, 17 μg/ml of aprotinin (ab146286, Abcam)) which polymerized in the defect. Then, the soft tissue was closed with stitches.

### Muscle Histology

Rectus femoris muscle tissue was collected at the end of day 10 time point. Injured and contralateral healthy hind limbs were removed at the pubic symphysis and skinned. Both limbs were then placed in 40 ml of 10% neutral buffered formalin for 48 h at room temperature to fix the soft tissue. Following fixation, the defect area was isolated by removing the hip joint and tibia; preserving the quadricep muscles spanning from the patella across the length of the femur. The legs were whole mounted, and paraffin sectioned (4 μm) longitudinally across the middle of the patella indicating mid-point of defect. Sections were stained with standard histological procedures for Mason's Trichrome and imaged using the Arperio slide scanner (Leica Biosystems, USA).

### MicroCT

Muscle regeneration was evaluated 10 days post-treatment by microCT as a percentage of muscle volume against contralateral healthy legs. Briefly, both hind legs were scanned with microCT with the Nucline NanoScan 3.04.020.0000 (RS 2 D) operated to retrieve 480 projections operating at energy of 50 kVp/670 μA with exposure time set to 300 ms. Scans were performed at maximum zoom with a zigzag motion. Reconstructed scans in 3D were analysed with a licensed PMOD imaging software v4.4 (PMOD Technologies LLC). Quantification of muscle volume was performed by creating a 7 mm×7 mm×7 mm ROI aligned along the length of the femur and at the edge of the patella. A threshold of −350 to 800 was applied and volume was measured within the designated ROI. Volume of muscle defect was calculated over volume of healthy contralateral muscle to generate a percentage of muscle volume restored.

### Human Satellite Cell Source and Culture

Normal human skeletal muscle satellite cells were purchased from Lifeline Cell Technology. Cells were thawed in a 37° C. waterbath and plated in pre-warmed StemLife™ Sk complete medium supplemented with antimicrobial (Gentamicin 30 mg/ml, Amphotericin B 15 μg/ml at a density of 5,000 cells per cm². Seeded cells were incubated at 37° C., 5% CO₂ for 24 h and refed to remove cryopreservation reagents. Cells reaching 70% confluency and actively proliferating were passaged or used for the following proliferation assay. To passage cell, aspirate medium and rinse with PBS. To detach cells, TrypLE™ Express Enzyme (12604021, Gibco™) was added into the culture vessel and incubated at 37° C., 5% CO₂ for 3 min to release the cells. Cells were detached by gently agitation and Trypsin was neutralised with equal volumes of PBS, collected by centrifugation at 150×g for 5 min for passaging or in vitro proliferation assay.

### Human Endothelial Cell Source and Culture Methods

Human umbilical vein endothelial cells (HUVECs) pooled were purchased from Cell Applications, Inc (200P-05N, Sigma-Aldrich). Cells were thawed in a 37° C. waterbath and plated in pre-warmed endothelial cell growth medium (211-500, Sigma-Aldrich). Seeded cells were incubated at 37° C., 5% CO₂ for 24 h and refed to remove cryopreservation reagents. Media was refed every other day until cells reach 60% confluency and passaged when cell density reaches 80% confluency. To passage cell, aspirate medium and rinse with PBS. To detach cells, TrypLE™ Express Enzyme (12604021, Gibco™) was added into the culture vessel and incubated for 1 minute at room temperature to release the cells. Cells were detached by gently agitation and Trypsin was neutralised with equal volumes of PBS, collected by centrifugation at 220× g for 5 min for passaging or in vitro proliferation assay.

### Production and Purification of the IgG1 Fusion Methods

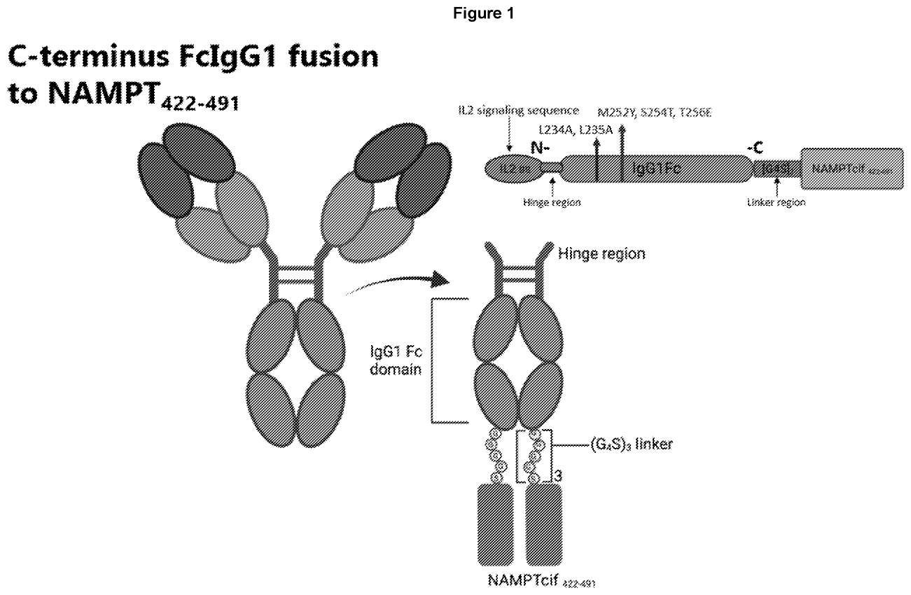

Human IgG1Fc NAMPTcif₄₂₂₋₄₉₁ fusion protein was designed according to the structure shown in FIG. 1. IgG1Fc NAMPT₄₂₂₋₄₉₁ gene was cloned into GeneArt expression ready vector pcDNA™3.4 TOPO™, a constitutive mammalian expression vector which provides high yield transfections. The vector is 6994 bps long and consists of a CMV promoter, a Woodchuck post-translational regulatory expression (WPRE) downstream to the cloning site to enhance transcript expression and a pUC origin to ensure high copy plasmid replication. The IgG1Fc NAMPT₄₂₂₋₄₉₁ gene is 984 bps long spanning from nucleotide 742 to 1725 and flanked by XBaI restriction site at the 5′ end and an AgeI restriction site at the 3′ end of the design construct. The recombinant pcDNA™3.4 TOPO™-IgG1FcNAMPT₄₂₂₋₄₉₁ was transformed into E. Coli DH5α competent cells (EC0111, Thermo Scientific) according to manufacturer's instructions for amplification. The transfected bacteria were amplified in LB-media with 100 μg/ml ampicillin (A0166-5G, Sigma-Aldrich) overnight at 37° C. with 225 rpm agitation. Vectors were isolated from transformants by Purelink™ HiPure Plasmid Maxiprep Kit (K210007, Thermo Scientific) and was verified by restriction enzyme mapping.

Expression of pcDNA™3.4 TOPO™-IgG1FcNAMPT₄₂₂₋₄₉₁ Expression Vector in Expi293 Cells

Expi293 expression system (A14635, Thermo Fisher) was used to express the recombinant proteins. Cells were maintained according to manufacturer's instructions as described below. Briefly, Expi293 cells were cultured in Expi293 medium (A1435101, ThermoFisher) at 37° C./8% CO₂ with 125 rpm agitation with 80% humidity in ventilated capped flasks (Corning® Erlenmeyer sterile polycarbonate with 0.2 μm ventilated caps). The CO₂ Resistant Shakers and Packages (88881104 ThermoFisher) was used for the incubation and these conditions were maintained throughout the culture. For maintenance and subculturing, cells were seeded at a density of 0.4×10⁶ viable cells/ml for 3 days until they reached a density of 3-5×10⁶/ml.

Expression of pcDNA™3.4 TOPO™-IgG1FcNAMPT₄₂₂₋₄₉₁ Transfection in Expi293 Cells

For efficient transfection of vector, Expi 293 cells of at least 95% viability and had not reached a density of more than 3-5×10⁶/ml was used. One day prior to transfection, cells were seeded at 2.5-3×10⁶/ml and allowed to grow overnight. On the day of transfection, cell viability was determined and cells were diluted to 3×10⁶ viable cells/ml with fresh pre-warmed Expi293 medium. For transfection, 1 μg/ml of total plasmid DNA in Opti-MEM medium (31985062, ThermoScientific) was added to the culture supplemented with ExpiFectamine™ 293 Reagent with Opti-MEM medium. Following a 22 h incubation ExpiFectamine™ 293 Transfection Enhancer 1 and ExpiFectamine™ 293 Transfection Enhancer 2 to the transfection flask. Cells were immediately returned to the incubator for 5 days at 37° C./8% CO₂ with 125 rpm agitation with 80% humidity. Supernatant (100 μl) aliquots were collected at days 1, 3 and 4 to verify the presence of Fc-NAMPT protein via SDS PAGE prior to final collection.

### Harvest of Transfected Cells

Five days after transfection, cells were harvested and centrifuged at 4000×g for 20 min and filtered through a 0.22 μm filter (CLS431096, Corning) and stored at −80° C. until purification.

### Purification

NAMPT₄₂₂₋₄₉₁ with and IgG1Fc domain can be readily purified with a HiTrap Protein A HP 5 ml column (GE17-0403-01, Cytiva). To purify the protein, the filtered supernatant was loaded into the HiTrap Protein A column attached to an ÄKTA START SYSTEM (29022094, Cytiva). The proteins were bound according to protein A column protocol with 20 mM sodium phosphate, pH 7.0 and eluted with 0.1M citric acid, pH 4.0. The elution was monitored by measuring the absorbance at 280 nm. Following purification, the buffer was exchanged twice by dialysis in a Slide-A-Lyzer G2 dialysis cassette with a 10K MW cut-off (87731, ThermoFisher). The proteins were concentrated with Amicon ultra centrifugal filters (UFC900308, Merck) with a 10 kDa MW cut-off. The concentrated protein was then filtered through a 0.22 micron syringe filter. Protein concentration was measured with a standard Pierce BCA Protein assay kit (23235, ThermoFisher) and corrected with extinction coefficient calculated from the Expasy protein parameter software. The purified proteins were stored in low-bind tubes at −80° C.

### Protein Characterisation

Purified proteins were verified by size through a reducing SDS-PAGE in 4-20% Mini-PROTEAN® TGX™ Precast Protein gels (4561093DC, BioRad) and sent for proteomics to confirm the size and amino acid sequence. Typical protein yield post purification of up to 1 mg/l can be obtained.

### Proliferation Assay Methods for Human Satellite Cells

Human skeletal muscle satellite cells were plated until a confluency of 70% and replated at 2,000 cells/well in a 96 well-flat bottom plate in pre-warmed StemLife basal medium supplemented with StemLife Sk LifeFactors (LS-1103, LifeLine Cell Technologies). Cells were then treated with 20 nM of equimolar proteins and cultured for 48 h at 37° C./5% CO₂. At the end of the timepoint, the microplate was gently inverted and blotted onto paper towels to remove medium from the wells and frozen at −80° C. For quantification, the plates were thawed and room temperature and cell proliferation were measured using the CyQUANT™ Cell Proliferation Assay Kit (C7026, ThermoFisher). Fluoresence was measured on a fluorescence micro-plate reader at 480 nm.

### Proliferation Assay Methods for Human Endothelial Cells

HUVEC cells at 70% confluency were pl replated at 2,000 cells/well in a 96 well-flat bottom plate in pre-warmed MCDB 131 medium supplemented (10372019, GIBCO). Cells were then treated with 20 nM of equimolar proteins and cultured for 48 h at 37° C./5% CO₂. At the end of the timepoint, the microplate was gently inverted and blotted onto paper towels to remove medium from the wells and frozen at −80° C. For quantification, the plates were thawed and room temperature and cell proliferation were measured using the CyQUANT™ Cell Proliferation Assay Kit (C7026, ThermoFisher). Fluoresence was measured on a fluorescence micro-plate reader at 480 nm.

### Larval Zebrafish Muscle Injury and EdU Pulse-Chase

Zebrafish larvae (4 dpf) were anaesthetized in 0.01% tricaine (MS-222) (Sigma-Aldrich) in Ringer's solution. Needle-stab injury was carried out in the dorsal myotome, consisting in a single 30-gauge needle puncture that generates an extensive injury with many damaged muscle fibres. NAMPT and IgG1-NAMPT treatments were carried out by incubating 4 dpf needle-stab-injured larvae in 57 nM of each treatment in Ringer's solution immediately after injury. Needle-stab-injured larvae at 6 dpf (2 dpi) were transferred into Ringer's solution containing 50 μg ml-1 EdU (Thermo Fisher Scientific) for 1 h and chased for a further 1.5 h before fixation. Samples were developed using the Click-iT EdU Alexa Fluor 647 imaging Kit (Thermo Fisher Scientific) following the manufacturer's protocol, followed by a phalloidin immunostaining (Thermo Fisher Scientific). EdU+ cells in a region encompassing two myotomes on either side of the injury were quantified as the number of EdU+ cells outside the injury region. EdU+ cells in the caudal haematopoietic tissue were excluded from this analysis. Statistical analysis has been conducted using Two-way ANOVA with Tuckey's multiple comparison test.

### Example 2—Human NAMPT Variants Stimulate Human Satellite Cell Proliferation

The inventors sought to compare the activity of IgG1-NAMPT₄₂₂₋₄₉₁ on human satellite cell proliferation. Human primary satellite cells were treated with 20 nM full IgG1-NAMPT₄₂₂₋₄₉₁ for 48 hours.

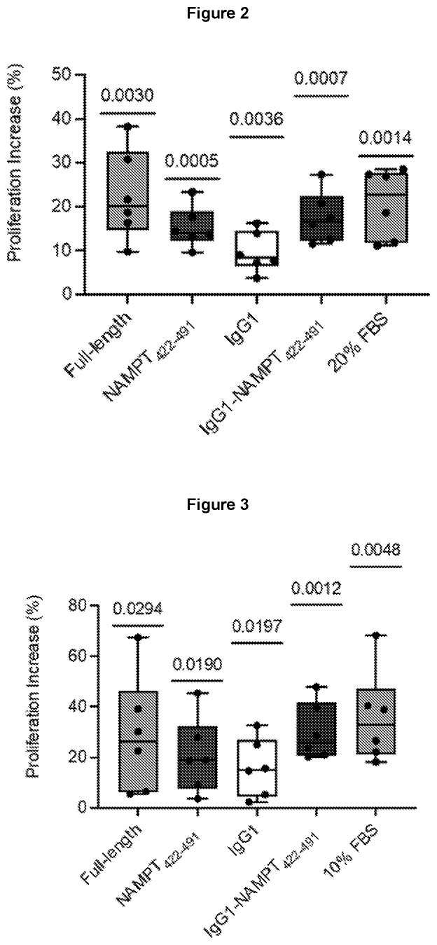

IgG1-NAMPT₄₂₂₋₄₉₁ exhibited increased satellite cell proliferation compared to PBS-negative control (FIG. 2).

### Example 4—Human NAMPT Variants Stimulate Human Endothelial Cell Proliferation

Similarly, the inventors next investigated the activity of IgG1-NAMPT₄₂₂₋₄₉₁ on human endothelial cell proliferation. Human endothelial cells were derived from umbilical vein and treated with IgG1-NAMPT₄₂₂₋₄₉₁ for 48 hours.

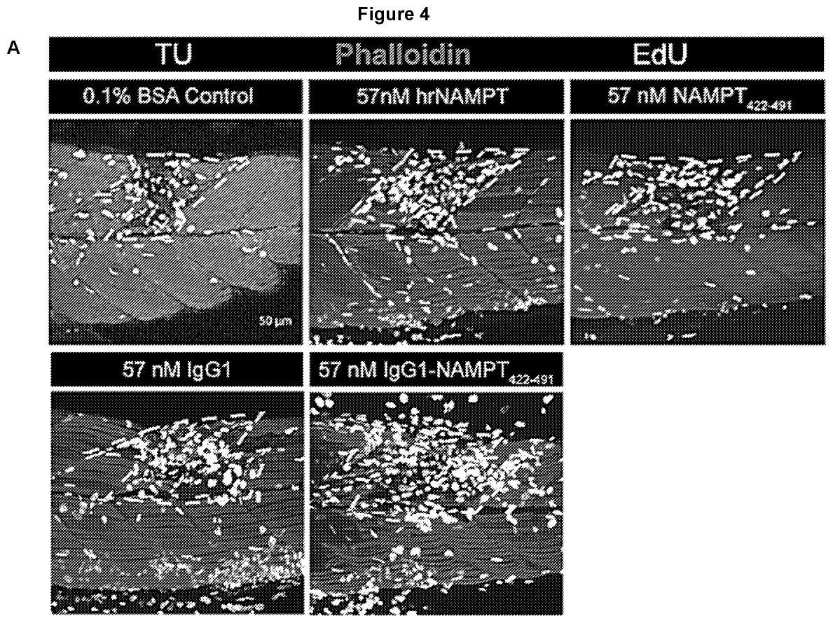

IgG1-NAMPT₄₂₂₋₄₉₁ displayed increased endothelial cell proliferation compared to PBS-negative control (FIG. 3).

### Example 5—Minimal Versions of NAMPT Protein Enhance Proliferation in Response to Muscle Injury in Zebrafish Larvae

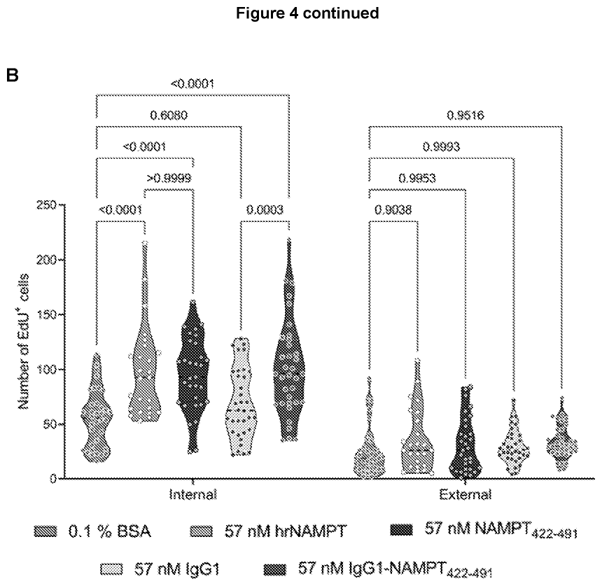

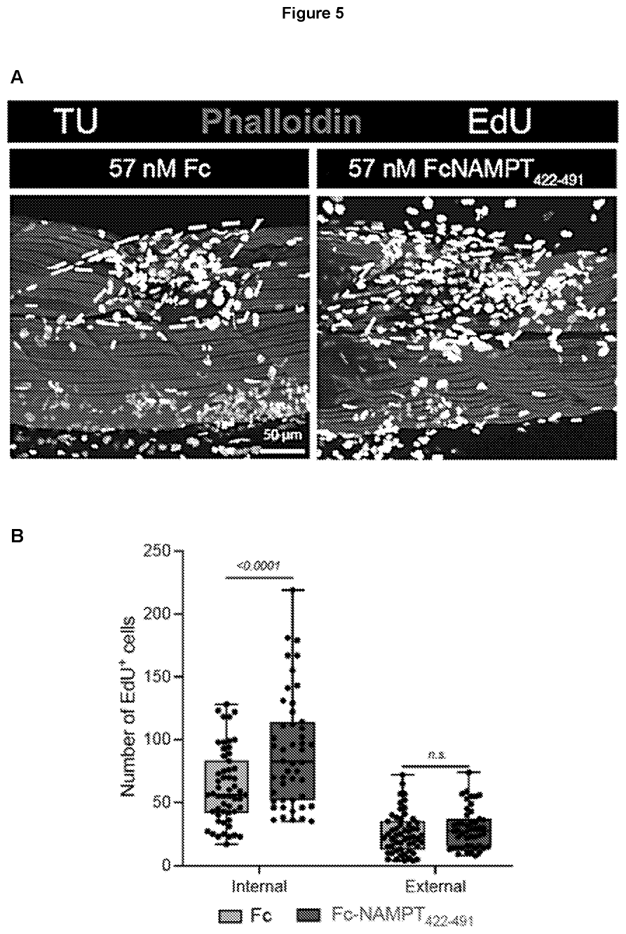

Next, the inventors tested the ability of IgG1-NAMPT₄₂₂₋₄₉₁ to stimulate cell proliferation in an in vivo zebrafish muscle injury response model. Treatment with IgG1-NAMPT₄₂₂₋₄₉₁ following needle-stick muscle injury to the zebrafish larvae induced a significant increase in cell proliferation within the injury zone compared to BSA negative or IgG1 alone controls (FIG. 4A-B & FIG. 5A-B). IgG1-NAMPT₄₂₂₋₄₉₁ did not stimulate cell proliferation external to the injury zone.

These findings indicate that IgG1-NAMPT₄₂₂₋₄₉₁ fusion protein stimulates muscle repair in the context of an acute injury of zebrafish larvae.

### Example 6—IgG1-NAMPT₄₂₂₋₄₉₁ Supplementation Accelerates Regeneration in a Mouse Model of Volumetric Muscle Loss

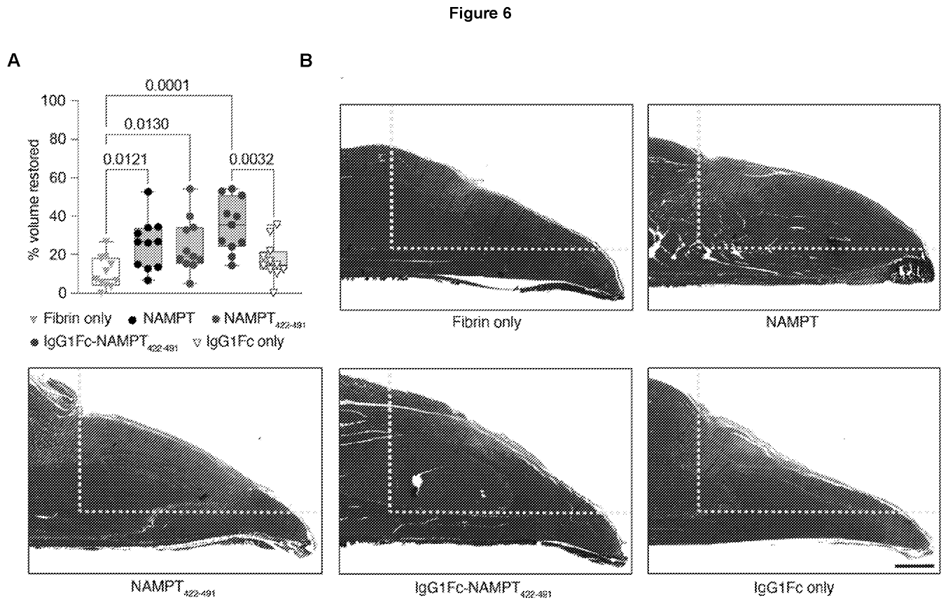

Volumetric muscle loss is an injury paradigm usually refractory to endogenous-stem cell mediated repair processes and is an area of unmet-clinical need. Here it is shown that the addition of IgG1-NAMPT₄₂₂₋₄₉₁ could accelerate regeneration in a mouse model of volumetric muscle loss. Strikingly, delivery of IgG1-NAMPT₄₂₂₋₄₉₁ into the muscle defect via a fibrin hydrogel, but not a fibrin only control hydrogel, was able to fully restore muscle architecture when applied to the wound site (FIG. 6A-B). The increase in muscle volume in mice treated with IgG1-NAMPT₄₂₂₋₄₉₁ was also greater than all other treatment groups, including NAMPT₄₂₂₋₄₉₁ without an IgG1 fusion.

Consistent with the results from the acute injury of zebrafish larvae, these findings indicate that IgG1-NAMPT₄₂₂₋₄₉₁ fusion protein stimulates muscle repair in the context of an acute injury of adult mammalian muscle.

### Example 7—Non-Clinical Single and Repeated Dose Toxicity Study with IgG1-NAMPT₄₂₂₋₄₉₁

### Objective

This study assessed the toxicity and tolerability of IgG1-NAMPT₄₂₂₋₄₉₁ treatment following a single intravenous administration and also following daily intravenous administration for a period of seven days in adult C57BL/6J mice.

### Study Design

### Treatment Groups

Groups of 5-6 weeks of age C57Bl/6J male and female mice were treated on Study day 1 with IgG1-NAMPT₄₂₂₋₄₉₁ by the intravenous route as either a single bolus dose of 1, 3, 7.5 and 10 μg/mouse (“single dose study”) or as a daily bolus dose at dose levels of 1, 3 and 10 μg/mouse over a period of seven days (“repeated dose study”). The repeated dose study included a vehicle control group of 0.9% saline. The treatment groups for the single dose phase and repeated dose phase are provided in Tables 4 and 5 below.

TABLE 4
Dose formulation - Single Dose Study

| | | Formulation | Volume | Dose Rate |
|---|---|---|---|---|
| | | Concentration | per Dose | (μg/ |
| Group | Treatment | (μg/mL) | (μL) | mouse) |
| 1 | IgG1-NAMPT₄₂₂₋₄₉₁ | 10 | 100 | 1 |
| 2 | IgG1-NAMPT₄₂₂₋₄₉₁ | 30 | 100 | 3 |
| 3 | IgG1-NAMPT₄₂₂₋₄₉₁ | 75 | 100 | 7.5 |
| 4 | IgG1-NAMPT₄₂₂₋₄₉₁ | 100 | 100 | 10 |

TABLE 5
Dose formulation - Repeated Dose Study

| | | Formulation | Volume | Dose Rate |
|---|---|---|---|---|
| | | Concentration | per Dose | (μg/ |
| Group | Treatment | (μg/mL) | (μL) | mouse) |
| 1 | Vehicle | Nil | 100 | 0 |
| 2 | IgG1-NAMPT₄₂₂₋₄₉₁ | 10 | 100 | 1 |
| 3 | IgG1-NAMPT₄₂₂₋₄₉₁ | 30 | 100 | 3 |
| 4 | IgG1-NAMPT₄₂₂₋₄₉₁ | 100 | 100 | 10 |

### Observations

Morbidity and mortality observations were recorded at least once daily during the at least 5 day acclimation period prior to dose administration an during Study Day 1 through to Study Day 8.

Clinical observations were conducted at least once during the acclimation period and at least once daily from Study Day 1 up to and including the day prior to termination (Study Day 7).

For the single dose study, on the day of each treatment, the observations were completed at approximately 5 min, 30 min, 60 min, and 4 h (±10 min) post-dose.

For the repeated dose study, observations were completed at approximately 30 min (±10 min) post-dose on Study Days 1-7.

The clinical observations included examination of animals for changes in skin and fur, eyes and mucous membranes, respiratory and circulatory function, gait and posture, behaviour, tremors or convulsion and any other abnormal findings. This also included daily examination of the tail for injection site reactions. Any other injection site lesions such as eschar, necrosis and ulceration were also noted.

### Body Weights

Body weights for all animals were recorded during the acclimatization phase and daily thereafter.

### Necropsy

On Study Day 8, all mice were subjected to a final body weight measurements and then euthanised. The animals were subjected to gross necropsy with a detailed examination of the external appearance of the animal and internal organs. All macroscopic observations were recorded with collection of any tissues with visible lesions into 10% neutral buffered formalin.

### Clinical Pathology—Repeated Dose Study

The mice were fasted for at least 4 h prior to terminal bleed sampling with access to food withdrawn but water available. After administration of an overdose of pentobarbitone to anaesthetise the animal, blood sample were collected puncture of the inferior vena cava.

The blood sample from all female mice (n=3) per dose group were used for haematology analyses. ~0.6 mL of blood was transferred into a K₃-EDTA tube and stored at 2-8° C. K₃-EDTA treated whole blood samples were analysed using an IDEXX ProCyte Haematology Analyser according to Test Facility standard operating procedures within 24 h of collection:

The blood sample from all male mice (n=3) per dose group were used for biochemistry analyses. ~0.6 mL of blood was transferred into a serum separator tube and subsequently processed to serum and the resulting sample transferred to a receiving tube and stored at 2-8° C. Serum sample were analysed using an IDEXX Catalyst Clinical Chemistry Analyser according to Test Facility standard operating procedures within 24 h of collection.

### Results

In the single dose study, there were no findings of morbidity or mortality nor were there any treatment-related clinical observations (data not shown). There were also no findings of changes in body weight (data not shown). Doses of 1, 3, 7.5 and 10 μg/mouse were tolerated when administered as a single bolus by the intravenous route.

In the repeat dose study, daily treatment with IgG1-NAMPT₄₂₂₋₄₉₁ was tolerated at dose levels of 1, 3 and 10 μg/mouse. Similar to the single dose study, there were no findings of morbidity or mortality nor were there any treatment-related clinical observations (data not shown). There were also no findings of changes in body weight or changes in terminal haematology or clinical biochemistry parameters which were considered related to treatment (data not shown).

In conclusion, this study suggests that a single dose treatment and daily treatment IgG1-NAMPT₄₂₂₋₄₉₁ for seven days is well-tolerated when intravenously administered to mice.

1. A fusion protein comprising:
(i) a NAMPT cytokine finger (cif) polypeptide; and
(ii) an Fc region of an antibody.

2. The fusion protein of claim 1, wherein the NAMPT cif polypeptide and Fc region of an antibody are joined directly or via a linker sequence.

3. The fusion protein of claim 1 or 2, wherein the N-terminus of the NAMPT cif polypeptide is joined directly or via a linker sequence to the C-terminus of the Fc region of an antibody.

4. The fusion protein of claim 1 or 2, wherein the C-terminus of the NAMPT cif polypeptide is joined directly or via a linker sequence to the N-terminus of the Fc region of an antibody.

5. The fusion protein of claim 1 or 2, wherein the fusion protein comprises a first NAMPT cif polypeptide that is N-terminal to the Fc region of the antibody and a second NAMPT cif polypeptide that is C-terminal to the Fc region of the antibody.

6. The fusion protein of any one of claims 1 to 5, wherein the NAMPT cif polypeptide comprises, consists essentially of or consists of an amino acid sequence of full length NAMPT cif, preferably the amino acid sequence shown in SEQ ID NO: 1.

7. The fusion protein of any one of claims 1 to 5, wherein the NAMPT cif polypeptide comprises an amino acid sequence of truncated NAMPT cif.

8. The fusion protein of any one of claim 7, wherein the truncation of NAMPT cif polypeptide is a N-terminal truncation.

9. The fusion protein of claim 8, wherein the N-terminal truncation is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids.

10. The fusion protein of claim 8, wherein the truncation is of the N-terminal residues 1-12 of the amino acid sequence of, or equivalent to, SEQ ID NO: 1.

11. The fusion protein of claim 8, wherein the truncation is of the N-terminal residues 1-20 of the amino acid sequence of, or equivalent to, SEQ ID NO: 1.

12. The fusion protein of claim 8, wherein the truncation is of the N-terminal residues 1-28 of the amino acid sequence of, or equivalent to, SEQ ID NO: 1.

13. The fusion protein of claim 8, wherein the truncation is of the N-terminal residues 1-35 of the amino acid sequence of, or equivalent to, SEQ ID NO: 1.

14. The fusion protein of any one or claims 1 to 13, wherein the NAMPT cif polypeptide is equal to, or less than, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, or 56 amino acids in length.

15. The fusion protein of any one or claims 1 to 14, wherein the NAMPT cif polypeptide comprises an amino acid sequence of any one of SEQ ID Nos: 1, 2, 3, 4, 5, 6, 7, 8, 9, 10 or 11 or comprises an amino acid sequence having 1, 2, 3, 4, 5, 6, 7 or 8 conservative or non-conservative amino acid substitutions, deletions or additions to the above sequences, and retains CCR5 or tissue stem cell interacting activity.

16. The fusion protein of any one or claims 1 to 14, wherein the NAMPT cif polypeptide consists of an amino acid sequence of any one of SEQ ID Nos: 1, 2, 3, 4, 5, 6, 7, 8, 9, 10 or 11 or consists of an amino acid sequence having 1, 2, 3, 4, 5, 6, 7 or 8 conservative or non-conservative amino acid substitutions, deletions or additions to the above sequences, and retains CCR5 or tissue stem cell interacting activity.

17. The fusion protein of any one or claims 1 to 14, wherein the NAMPT cif polypeptide consists of an amino acid sequence having equal to, or at least, 85% sequence identity to the amino acid sequence of SEQ ID NO: 1, preferably the amino acid sequence of the polypeptide is equal to, or at least, 86%, 87%, 88%, 89%, 90%, 91%, 92%, 93%, 94%, 95%, 96%, 97%, 98%, or 99% identical to SEQ ID NO: 1.

18. The fusion protein of any one or claims 1 to 14, wherein the NAMPT cif polypeptide consists of an amino acid sequence having equal to, or at least, 85% sequence identity to the amino acid sequence of SEQ ID NO: 4 or 5, preferably, the amino acid sequence of the polypeptide is equal to, or at least, 86%, 87%, 88%, 89%, 90%, 91%, 92%, 93%, 94%, 95%, 96%, 97%, 98%, or 99% identical to SEQ ID NO: 4 or 5.

19. The fusion protein of any one or claims 1 to 14, wherein the NAMPT cif polypeptide consists of an amino acid sequence having equal to, or at least, 85% sequence identity to the amino acid sequence of SEQ ID NO: 6 or 7, preferably, the amino acid sequence of the polypeptide is equal to, or at least, 86%, 87%, 88%, 89%, 90%, 91%, 92%, 93%, 94%, 95%, 96%, 97%, 98%, or 99% identical to SEQ ID NO: 6 or 7.

20. The fusion protein of any one or claims 1 to 14, wherein the NAMPT cif polypeptide consists of an amino acid sequence having equal to, or at least, 85% sequence identity to the amino acid sequence of SEQ ID NO: 8 or 9, preferably, the amino acid sequence of the polypeptide is equal to, or at least, 86%, 87%, 88%, 89%, 90%, 91%, 92%, 93%, 94%, 95%, 96%, 97%, 98%, or 99% identical to SEQ ID NO: 8 or 9.

21. The fusion protein of any one or claims 1 to 14, wherein the NAMPT cif polypeptide consists of an amino acid sequence having equal to, or at least, 85% sequence identity to the amino acid sequence of SEQ ID NO: 10 or 11, preferably, the amino acid sequence of the polypeptide is equal to, or at least, 86%, 87%, 88%, 89%, 90%, 91%, 92%, 93%, 94%, 95%, 96%, 97%, 98%, or 99% identical to SEQ ID NO: 10 or 11.

22. The fusion protein of any one or claims 1 to 14, wherein the NAMPT cif polypeptide consists of an amino acid sequence of SEQ ID NO: 1.

23. The fusion protein of any one or claims 1 to 14, wherein the NAMPT cif polypeptide consists of an amino acid sequence of SEQ ID NO: 4.

24. The fusion protein of any one or claims 1 to 14, the NAMPT cif polypeptide consists of an amino acid sequence of SEQ ID NO: 6.

25. The fusion protein of any one or claims 1 to 14, wherein the NAMPT cif polypeptide consists of an amino acid sequence of SEQ ID NO: 8.

26. The fusion protein of any one or claims 1 to 14, wherein the NAMPT cif polypeptide consists of an amino acid sequence of SEQ ID NO: 10.

27. The fusion protein of any one of claims 1 to 26, wherein the Fc region of an antibody is an Fc region of an IgG, IgA, IgD, IgE, or IgM, preferably the Fc region is from an IgG antibody, such as an IgG1, an IgG2, an IgG2b, an IgG3 or an IgG4 antibody.

28. The fusion protein of any of one of claims 1 to 27, wherein the Fc comprises two heavy chain fragments, preferably the CH2 and CH3 domains of said heavy chain.

29. The fusion protein of any of claims 1 to 28, wherein the Fc region does not comprise one or more of a VH, VL, light chain CH1 and heavy chain CH1.

30. The fusion protein of any of claims 1 to 29, wherein the Fc region comprises, consists essentially of or consists of a hinge region, CH2 and CH3 domain.

31. The fusion protein of any one of claims 1 to 30, wherein the Fc region comprises one or more amino acid substitutions for reducing affinity for the Fc receptor (FcR, including any of FcγRI, FcγRII and FcγRIII) compared to wild-type or naturally occurring Fc regions.

32. The fusion protein of any one of claims 1 to 31, wherein the fusion protein has an affinity for FcR that is less than about 250 nM, preferably less than 500 nM, less than 1000 nM, most preferably less than 2000 nM.

33. The fusion protein of any one of claims 1 to 32, wherein the Fc region comprises:
(a) L234A and L235A; and/or
(b) D265A and N297A (according to EU numbering of residues).

34. The fusion protein of any one of claims 1 to 33, wherein the Fc region comprises M252Y, S254T and T256E.

35. The fusion protein of any of claims 1 to 34, wherein the Fc region of an antibody comprises, consists essentially of or consists of an amino acid sequence as shown in SEQ ID NOs: 38 or 40.

36. The fusion protein of any of claims 1 to 35, wherein the fusion protein comprises a linker between the NAMPT cif polypeptide and the Fc region of an antibody

37. The fusion protein of any one of claims 1 to 36, wherein the fusion protein comprises a linker between the N-terminus of the Fc regions and the C-terminus of the NAMPT polypeptide.

38. The fusion protein of any one of claims 1 to 36, wherein the fusion protein comprises a linker between the C-terminus of the Fc regions and the N-terminus of the NAMPT polypeptide.

39. The fusion protein of any one of claims 36 to 38, wherein the linker is a peptide linker.

40. The fusion protein of claim 39, wherein the peptide linker comprises the amino acid sequence Gly-Gly-Ser (GGS), Gly-Gly-Gly-Ser (GGGS) or Gly-Gly-Gly-Gly-Ser (GGGGS).

41. The fusion protein of any one of claims 39 to 40, wherein the linker comprises or consists of the sequence shown in SEQ ID NO: 41.

42. The fusion protein of any one of claims 1 to 41, wherein the fusion protein further comprises a signal sequence.

43. The fusion protein of claim 42, wherein the signal sequence is an IL-2 signal sequence.

44. The fusion protein of claim 43, wherein the signal sequence consists of the amino acid sequence as shown in SEQ ID NO: 36

45. The fusion protein of any one of claims 1 to 44, wherein the fusion protein comprises an amino acid sequence as shown in any one of SEQ ID NOs: 42 to 61.

46. The fusion protein of any one of claims 1 to 45, wherein the polypeptide is monomeric form.

47. The fusion protein of any one of claims 1 to 45, wherein the polypeptide is dimeric form, wherein each monomer comprises:
(i) a NAMPT cytokine finger (cif) polypeptide; and
(ii) an Fc region of an antibody.

48. A nucleic acid encoding the fusion protein of any one of claims 1 to 47.

49. A vector comprising the nucleic acid of claim 48.

50. A cell comprising a nucleic acid of claim 48, or a vector of claim 49, preferably the cell is a macrophage or muscle satellite cell.

51. A composition comprising a fusion protein, nucleic acid, vector, or cell of any one of the preceding claims and a pharmaceutically acceptable carrier, diluent or excipient.

52. A method of stimulating proliferation of a satellite cell, the method comprising administering to a cell or subject an effective amount of a fusion protein, nucleic acid, vector, cell or composition of any one of the preceding claims, thereby stimulating proliferation of a stem cell.

53. A method of stimulating muscle tissue regeneration in a subject, the method comprising administering to a muscle of the subject an effective amount of a fusion protein, nucleic acid, vector, cell or composition of any one of the preceding claims, thereby stimulating muscle tissue regeneration.

54. A method of stimulating muscle tissue regeneration in a subject where inflammation is undesirable, the method comprising administering to a muscle an effective amount of a fusion protein, nucleic acid, vector, cell or composition of any one of the preceding claims, thereby stimulating muscle tissue regeneration in this subject.

55. The method of claim 54, wherein the inflammation that is undesirable is inflammation mediated by TLR activation, preferably TLR4 activation.

56. The method of claim 54 or 55, wherein the subject has been diagnosed with an inflammatory myopathy.

57. The method of claim 56, wherein the inflammatory myopathies is polymyositis, dermatomyositis, inclusion body myositis, or necrotizing autoimmune myopathy.

58. A method of treating an inflammatory myopathy in a subject, the method comprising administering to a muscle of the subject an effective amount of a fusion protein, nucleic acid, vector, cell or composition or cell of any one of the preceding claims, thereby treating an inflammatory myopathy.

59. The method of claim 58, wherein the inflammatory myopathy is polymyositis, dermatomyositis, inclusion body myositis or necrotizing autoimmune myopathy.
